(19) United States
(12) Patent Application Publication
(10) Pub. No.: US 2015/0352100 A1
(43) Pub. Date: Dec. 10, 2015

(54) USE OF SELECTIVE DELTA-OPIOID RECEPTOR ANTAGONISTS AND SPECIFIC SENSORY RECEPTOR LIGANDS

(71) Applicant: AVANT DERMA PTE. LTD., Singapore (SG)

(72) Inventors: Paul Bigliardi, Sentosa (SG); Mei Bigliardi, Sentosa (SG)

(21) Appl. No.: 14/762,046

(22) PCT Filed: Jan. 21, 2014
(86) PCT No.: PCT/SG2014/000025

§ 371 Date: Jul. 20, 2015

Related U.S. Application Data
(60) Provisional application No. 61/754,756, filed on Jan. 21, 2013.

(51) Int. Cl. A61K 31/485; A61K 31/4748; A61K 45/06; A61K 8/49; A61Q 19/08; A61K 31/439; A61K 31/4745

(52) U.S. Cl. CPC A61K 31/485; A61K 31/439; A61K 31/4748; A61K 31/4745; A61K 8/4926; A61Q 19/08; A61K 45/06; A61K 2800/20

(57) ABSTRACT

The present invention relates to a compound or combination for use in the treatment of skin wounds, skin aging, skin tumors and/or skin sensation conditions and/or for treatment to improve skin repair, wherein the compound or combination is:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor, and
   wherein the treatment comprises a step of administering an effective amount of the compound or combination to a subject in need of such treatment.

### FIELD OF THE INVENTION

The invention relates to the fields of epithelial pharmacology and more specifically to a method for treating epithelial conditions by using an effective amount of a selective delta-opioid receptor antagonist and/or a specific ligand for sensory receptor.

### BACKGROUND

Functioning to protect the body from various external threats and being in contact with various external stimuli, the epithelium is prone to sensation, wounds and aging. This includes all types of epithelia in respiratory and gastrointestinal system, eye, mucosal epithelia (oral, anal, uro-genital) and the largest and most visible, prominent, exposed and accessible epithelia of the skin with its appendages (e.g. hair, sebaceous and sweat glands and nails).

The human skin consists of two layers, the bottom thicker layer (dermis and subcutis) and the top thinner layer (epidermis). The dermis is the layer which provides strength, elasticity, thickness to the skin and blood supply by a dense network of capillaries with endothelial cells and subcutaneous tissue which consists mainly of fat tissue with adipocytes. The main cell type of the dermis is the fibroblast, which is responsible for synthesis and secretion of all the dermal matrix components such as collagen, elastin, and glycosaminoglycans. Collagen provides the strength; elastin is responsible for the elasticity, and glycosaminoglycans afford the skin moistness and plumpness. With aging, the thickness of the dermal layer is reduced and this is believed to be partially responsible for the formation of wrinkles in aging skin. The top layer of human skin, or the epidermis, which provides the resilience and the barrier properties of the skin, is composed of many different cell types including keratinocytes, melanocytes, Langerhans and Merkel cells. In addition, the dermis with the fibroblasts, the connective tissue and the blood vessels, interacts directly with the epithelial cells, determining the quality of the entire skin, including epidermis.

Pleasant and unpleasant epithelium sensations (relaxed, sense of well-being, pain, burning, itch, tingling), epithelium wounds and skin aging conditions (wrinkling, pigmentations, atrophy) are treated with a variety of different topical and/or systemic medications and/or cosmetic compositions. One common approach for improving the appearance of skin has been through the stimulation of proliferating of new skin cells (keratinocytes, fibroblasts). Consequently, the skin takes on a younger appearance as these new skin cells do provide more structure and retain more moisture. There is still requirements for the cosmetics, cosmeceuticals and therapy of damaged epithelium and surrounding structure including cutaneous, respiratory, gastro-intestinal and mucosal epithelia, in particular in patho-physiology of wound healing, peripheral sensation, ageing conditions, as well as tumor growth/invasiveness, autoimmune and inflammatory disorders. In other words, there is still a need for cosmetics, cosmeceuticals, topical medications and methods of therapy for damaged epithelial, associated and surrounding structures including cutaneous, respiratory, gastro-intestinal and mucosal epithelia, in particular in patho-physiology of wound healing, peripheral sensation, ageing conditions, as well as to treat and to reduce the effects of tumor growth/invasiveness, autoimmune and inflammatory disorders.

Opioid ligands and their receptors in the skin comprise part of the endogenous opioid system that includes peptides such as enkephalins, endorphins, dynorphins and endomorphins and three opioid receptors commonly designated as delta (δ) (DOR), kappa (κ) (KOR) and mu (μ) (MOR). Several review articles elucidated the presence of opioid receptors, and the endogenous peptides and other compounds with which they interact (Akil et al., Annu. Rev. Neurosci. 7:223-255 (1984); Paul L. Bigliardi et al, “Opioids and the skin—where do we stand?”; Experimental Dermatology; 18:424-430 (2009)). These three opioid receptors (δ, μ, κ) are involved in the modulation of a variety of opioid effects.

There is growing evidence that opioid receptors (OR) and their endogenous opioid agonists are expressed and released in different skin structures. These include peripheral nerve fibres, keratinocytes, fibroblasts, melanocytes, hair follicles, sebocytes, endothelial cells and immune cells and adipocytes. For example it has been reported that delta-opioid receptors (DOR) are expressed in human skin (Bigliardi-Qi M. et al; “Deletion of delta-opioid receptor in mice alters skin differentiation and delays wound healing”; Differentiation 2006: 74: 174-185) and that DOR are highly up-regulated in the skin of fibromyalgia patients (Salemi S. et al; “Up-regulation of delta-opioid receptors and kappa-opioid receptors in the skin of fibromyalgia patients”; Arthritis Rheum; 56: 2464-2466 (2007)).

Significant atrophy of the epidermis in MOR and DOR knockout mice has been also reported, suggesting a controlling role for MOR and DOR on keratinocyte differentiation. DOR knockout mice also up-regulated the expression of the keratinocyte differentiation marker cytokeratin 10 and healed wounds more slowly. DOR KO mice showed a greater increase in epidermal thickness during wound healing, compared to wild-type mice at day 3. These results suggest a role for DOR in keratinocyte migration as well as differentiation (Paul L. Bigliardi et al; vide supra). DOR influences cell differentiation, migration, adhesion, cytokeratin and cytokine expression and regulation of metalloproteinases and connective tissue in skin (Bigliardi et al, Differentiation, 2006).

In the light of the above-mentioned results, selective AGONISTS for the DOR have shown promising therapeutic potential as they can stimulate the activity of these receptors. For example selective AGONISTS have been reported as analgesics without the adverse side effects associated with morphine and other opioid drugs which are selective for the opioid receptor.

In addition, at the activation of DOR, taste receptors and olfactory receptors are activated and their ligands required for sensing dangerous signals in order to have immune response and cell functions to repair, regenerate and stimulate stem cells and high proliferative cell types e.g wound healing, hair follicle and tumor growth. The ligands of these receptors could be used to stimulate their specific receptors independently or conjunctionally with opioid receptor activation to cope with tissue damage and sensation loss.

Therefore, the development of an efficient topical treatment of skin with selective DOR ligands, taste receptor and olfactory receptor ligands independently or conjunctionally for the treatment of wounds, skin ageing and epithelial sensation disorder would be beneficial in the therapeutic and cosmetic fields. For example, there is a need for further effective cosmetic and/or therapeutic treatments for skin conditions using selective DOR ligands, taste receptor and olfactory receptor ligands, individually or in combination.

### SUMMARY OF THE INVENTION

To the contrary to what has been expected, the Applicants have surprisingly developed a topical treatment of skin with selective delta-opioid ANTAGONISTS for the treatment of epithelial conditions.

Accordingly, in a first aspect, the present invention provides a method for treating epithelial conditions by administering an effective amount of a selective delta-opioid receptor (DOR) antagonist to a subject in need thereof.

In one aspect, the present invention provides a method for treating skin wounds, skin aging, skin tumors and/or skin sensation conditions and/or for improving skin repair, comprising a step of administering an effective amount of:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor,
   to a subject in need of the treatment, as set out in claim 1. Embodiments of this aspect are set out in claims 2 to 38.

Preferably, the selective DOR antagonist is selected from the group consisting of naltrindole, methylnaltrindole, N,N(Me)₂-Dmt-Tic-OH, SoRI-9409 and naltriben, or pharmaceutically acceptable salts thereof. More preferably, the selective DOR antagonist is naltrindole or pharmaceutically acceptable salts thereof. In addition, the effects of delta-opioid receptor (DOR) and mu-opiate receptor (MOR) agonists in wound healing and skin sensation will be amplified and intensified by topical treatment with these selective DOR antagonists by using them in combined treatment.

The epithelial conditions comprise but are not limited to skin wounds, skin aging, skin tumors and/or skin sensation conditions. For example, the epithelial conditions comprise but are not limited to wounds, aging, tumors and/or sensation conditions. The term epithelial in this invention includes ALL various types of the barrier layers from respiratory and gastrointestinal system, eye, mucosal epithelia (oral, anal, uro-genital) and the largest and most visible, prominent, exposed and accessable epithelia of the skin with its appendages (e.g. hair, sebaceous and sweat glands and nails).

For example in skin, the epithelial wound is caused by burn, chemical and/or mechanical injury to the epithelium. The skin aging conditions are preferably selected from the group consisting of wrinkles, skin discoloration, rosacea, senile angiomas, vessel fragility with haematomas, photo-aging, lentigines, loss of elasticity, increased fragility of skin, dry and itchy skin and delayed wound healing. Preferably, the epithelial wound is caused by burn, chemical and/or mechanical injury to the epithelium. The skin aging conditions are preferably selected from the group consisting of wrinkles, skin discoloration/pigmentation, rosacea, senile angiomas, vessel fragility with haematomas, photo-aging, lentigines, loss of elasticity, increased fragility of skin, dry and itchy skin and delayed wound healing/repair.

Without being bound by theory, it is believed that opioids and their ligands are involved in all phases of wound healing, from the initial inflammation phase, to proliferation and re-epithelialisation phase to the final regeneration phase. The initial inflammation phase is a crucial part of wound healing and often involved in delayed wound healing. Skin tumors are also closely related to the processes related to wound healing and skin ageing or chronic skin damages. In other words, the development of skin tumors may also be associated with the processes relating to wound healing, skin ageing and/or chronic skin damage.

Therefore, the method of the present invention may also be applied to the subjects in need of treatment for inflammatory and/or autoimmune disorders.

In one aspect, the present invention provides a topical pharmaceutical composition for treating epithelial conditions, comprising at least one selective DOR antagonist and a pharmaceutically acceptable carrier. The pharmacological carrier or vehicle described in this invention may include all different types and variations of topical formulations (e.g. cream, ointment, powder, gel, liposomes etc.etc).

The topical pharmaceutical composition in vehicles of the present invention may further include one or more DOR agonist and/or one or more other pharmaceutically active ingredient selected from the group consisting of an antibacterial agent, an antiviral agent, an anti-fungal agent, an anti-parasitic agent, an anti-inflammatory agent, an analgesic agent and an anti-pruritic agent.

In one aspect, the present invention provides a method for stimulating differentiation and proliferation of various epithelial cells, comprising a step of contacting said cells with a selective DOR antagonist.

Said method may further comprise a step of contacting said epithelial cells with a selective DOR agonist. Said selective DOR agonist is selected from the group consisting of SNC-80, BW373U86, DPI-287 and DPI-3290.

Said epithelial cells comprise all types of epithelial cells, including but not limited to skin cells and cells from mucosal (oral, ano-genital), respiratory, gastro-intestinal, uro-genital, eye and ear epithelia. The skin cells comprise, such as keratinocytes, fibroblasts, melanocytes, Merkel cells, dendritic cells and other skin immune cells, adipocytes and cells in skin appendages, such as hair follicles, sebaceous and sweat glands and nails. In particular, the opioid antagonists can have additional value to treat disorders of skin appendages, especially hair and sebaceous gland disorders. For example, said epithelial cells comprise all types of epithelial cells, including but not limited to skin cells and cells from mucosal, respiratory, gastro-intestinal epithelia. The skin cells comprise, such as keratinocytes, fibroblast, melanocyte, dendritic cells and skin immune cells.

The method according to the present invention may be used for stimulating nerve regeneration and/or endothelial homeostasis.

In one aspect, the present invention provides a method for treating skin sensation conditions, by administering an effective amount of a selective ligand for sensory receptor to a subject in need thereof.

The sensory receptor is a taste receptor and/or an olfactory receptor, and is selected from the group consisting of taste receptor TAS2R14 and the olfactory receptors OR2T4 and OR11G2. The selective ligand for the sensory receptor is thujone or flufenamic acid (FFA). More particularly, the sensory receptor is a taste receptor and/or an olfactory receptor, and is selected from the group consisting of taste receptor TAS2R14 (TAS2R10) and various olfactory receptors (e.g. OR2T4). The selective ligands for the sensory receptor are thujone or flufenamic acid (FFA).

The method according to the fourth aspect of the present invention can be used in combination with the method according to the first or third aspect of the present invention.

Therefore, the present invention provides a method for treating epithelial conditions by administering an effective amount of a selective DOR antagonist and/or a selective ligand for sensory receptor to a subject in need thereof.

The present invention also provides a method for stimulating differentiation and proliferation of epithelial cells, comprising a step of contacting said cells with a selective DOR antagonist and/or a selective ligand for sensory receptor to a subject in need thereof.

In one aspect, the present invention provides a method for modulating differentiation and proliferation of cells, comprising a step of contacting said cells with a selective DOR antagonist and/or a selective ligand for a sensory receptor. Said differentiation and proliferation processes may be involved in skin homeostasis and wound healing. The method may thus comprise stimulating differentiation of the cells. The cells may be high-proliferative cells. The high-proliferative cell may be an epithelial cell or a stem cell, optionally wherein the stem cell is not derived from a human embryonic stem cell. The method may be an in vitro method. Both stem cells and keratinocytes are highly proliferating cells. However, stem cells are un-differentiated, proliferative cells and keratinocytes are differentiated, proliferative cells.

In a further aspect, the present invention provides a method for screening selective ligands for sensory receptor, comprising the steps of over-expressing a selective ligand for sensory receptor in epithelial cells, and screening the screening selective ligands for the sensory receptor.

A further aspect of the invention relates to a method for treating skin wounds, skin aging, skin tumors and/or skin sensation conditions and/or for treatment to improve skin repair, comprising a step of administering an effective amount of a selective delta-opioid receptor (DOR) antagonist or a combination of a selective DOR antagonist and an opioid receptor agonist, to a subject in need of the treatment, as set out in claim 1 below. Embodiments of this aspect are listed in claims 2 to 38.

A yet further aspect of the invention relates to a use of a compound or combination in the preparation of a medicament for the treatment of skin wounds, skin aging, skin tumors and/or skin sensation conditions and/or for treatment to improve skin repair, wherein the treatment comprises a step of administering an effective amount of the compound or combination to a subject in need of such treatment, wherein the compound or combination is:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor,
   as set out in claim 39 below. Embodiments of this aspect are listed in claims 40 to 76.

A yet further aspect of the invention relates to a compound or combination for use in the treatment of skin wounds, skin aging, skin tumors and/or skin sensation conditions and/or for treatment to improve skin repair, wherein the compound or combination is:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor, and
   wherein the treatment comprises a step of administering an effective amount of the compound or combination to a subject in need of such treatment, as set out in claim 77 below. Embodiments of this aspect are listed in claims 78 to 114.

A still further aspect of the invention relates to a topical pharmaceutical composition for treating disorders of skin appendages and pigmentation, skin wounds, skin aging, skin tumors and/or skin sensation conditions and/or for treatment to improve skin repair, comprising an effective amount of:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor,
   and a pharmaceutically acceptable diluent, adjuvent or, more particularly, carrier, as set out in claim 115. Embodiments of this aspect are listed in claims 116 to 145.

A yet still further aspect of the invention relates to a medical device for application of the topical pharmaceutical composition according to the aspect disclosed above, wherein the device is a dermal patch or a bandage comprising said topical pharmaceutical composition, as set out in claim 146. Embodiments of this aspect are listed in claim 147.

One aspect of the invention relates to a kit comprising:
(A) a first topical pharmaceutical composition comprising a selective DOR antagonist and a pharmaceutically acceptable adjuvant, diluent or carrier; and
(B) a second topical pharmaceutical composition comprising at least one pharmaceutical active ingredient and a pharmaceutically acceptable adjuvant, diluent or carrier, and optionally
(C) instructions for the simultaneous, concomitant or sequential administration of the selective DOR antagonist of (A) and the at least one pharmaceutical active ingredient of (B), to a subject in need thereof,
   wherein the at least one pharmaceutical active ingredient of (B) is selected from the group consisting of an anti-bacterial agent, an anti-viral agent, an anti-fungal agent, an anti-parasitic agent, an anti-inflammatory agent, an analgesic agent and an anti-pruritic agent, an opioid receptor agonist and a selective ligand for a sensory receptor, as set out in claim 148. Embodiments of this aspect are listed in claims 149 to 155.

In one aspect, the present invention provides a method for modulating differentiation and proliferation of cells, comprising a step of contacting said cells with a selective DOR antagonist and/or a selective ligand for a sensory receptor, as set out in claim 156. Embodiments of this aspect are listed in claims 157 to 183. The cells may be at least one high proliferative cell, e.g. epithelial cell or stem cell.

A still further aspect of the invention relates to a method for stimulating differentiation and proliferation of a high-proliferative cell, comprising a step of contacting said cells with a selective DOR antagonist and/or a selective ligand for a sensory receptor.

A further aspect of the invention relates to a cosmetic skin care composition comprising an effective amount of:
(a) at least one selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor,
   and a cosmetically acceptable adjuvant, diluent or, more particularly, carrier as set out in claim 184. Embodiments of this aspect are listed in claims 185 to 206.

A further aspect of the invention relates to a cosmetic skin care kit comprising:
(A) at least a first cosmetic skin care composition, the first cosmetic skin care composition comprising a selective DOR antagonist and a cosmetically acceptable adjuvant, diluent or carrier; and
(B) at least a second cosmetic skin care composition, the second cosmetic skin care composition comprising an opioid receptor agonist and/or a selective ligand for a sensory receptor, and a cosmetically acceptable adjuvant, diluent or carrier, and optionally
(C) instructions for the simultaneous, concomitant or sequential administration of the selective DOR antagonist of (A) and the opioid receptor agonist and/or the selective ligand for a sensory receptor of (B), to a subject in need thereof,
   as set out in claim 207. Embodiments of this aspect are listed in claims 208 to 213.

One aspect of the invention relates to a method for screening selective ligands for a sensory receptor, comprising the steps of over-expressing a sensory receptor in epithelial cells, and screening selective ligands for the sensory receptor, as set out in claim 214. Embodiments of this aspect are listed in claims 215 to 216.

### BRIEF DESCRIPTION OF THE FIGURES

FIG. 1 shows mean healing rate [wound diameter in mm²] of 6 mm punch wounds on the back of mice after topical application of Placebo (ointment only), 1% Naltrexone (general opioid receptor antagonist), 1% SNC 80 (specific DOR agonist), 1% Dalargin (enkephalin analogue) and 1% selective (specific) DOR antagonist Naltrindole. The selective DOR antagonist significantly improved wound healing at days 5 and 7 (Students T-test). In other words, an improvement in wound healing by 3 days was seen.

FIG. 2 shows the photos of the wound healing in mice treated with placebo cream vs. cream containing 1% Naltrindole. The mice treated with Naltrindol show macroscopically and microscopically a significant better healing than the mice treated with placebo cream or other DOR ligands.

FIG. 3 shows the epidermal DOR expression in 28 Chinese women from northern China in different age groups. The DOR expression was measured by immune-fluorescence using a specific anti-DOR antibody and DOR expression was significantly reduced in epidermis of older woman (50-70 years) compared to younger population. This down-regulation was independent from UV-induced photoaging (same trend in sun exposed as in sun protected skin areas) and shows a role of DOR in skin ageing.

FIG. 4 shows the correlation between photo-induced hyperpigmentations (Lentigines or taches) and epidermal DOR expression. The pigmentation was evaluated by blinded method by a specialist from photos taken from the arms of the volunteers. In conclusion, the more pigmentation is visible the lower is epidermal DOR expression. This suggests a role of DOR in skin pigmentation.

FIG. 5 shows the correlation between age related skin wrinkling [rides] and epidermal DOR expression. The wrinkling was evaluated with blinded method by specialists from photos from the arm of the volunteers. In conclusion, the more wrinkles are present the lower is epidermal DOR expression. This suggests a role of DOR in skin age related skin wrinkling.

FIG. 6 shows real time PCR of DOR-mRNA expression in cultured human primary keratinocytes. Incubation of keratinocytes with specific DOR antagonist Naltrindole and specific DOR agonist SNC-80 increases significantly the expression of DOR.

FIG. 7 shows real time PCR of DOR-mRNA expression in cultured human primary melanocytes. Incubation of human melanocytes with specific DOR antagonist Naltrindole and co-incubation of specific DOR antagonist and agonist SNC-80 increases significantly the expression of DOR.

FIG. 8 shows real time PCR of DOR-mRNA expression in cultured human primary fibroblasts. Incubation of fibroblasts with specific DOR antagonist Naltrindole increases significantly the expression of DOR.

FIG. 9 shows western blot analysis of DOR protein expression in primary human cultured keratinocytes. Exposure of keratinocytes to specific DOR antagonist Naltrindole and agonist SNC-80 up-regulates the DOR protein expression.

FIG. 10 shows western blot analysis of DOR protein expression in human cultured melanocytes. Exposure of melanocytes for 6 h to specific DOR antagonist Naltrindole, but not to specific agonist SNC-80 upregulates DOR expression. The combination of antagonist and agonist can up-regulate the protein as well, however there seems to exist a delicate balance between agonist and antagonist.

FIG. 11 shows western blot analysis of DOR protein expression in human cultured fibroblasts. The western blot analysis shows an important upregulation of DOR expression after 24 h exposure to specific DOR antagonist (Naltrindole) and agonists and some of their combinations.

FIGS. 12a-b: FIG. 12a is a picture of the western blot showing the stimulation of Erk phosphorylation in primary human keratinocytes by specific DOR antagonist Naltrindole (this effect was antagonized by specific DOR ligand SNC-80). FIG. 12b shows the quantification of the bands specific for Erk phosphorylation and it emphasizes the massive activation of Erk by the DOR antagonist Naltrindole.

FIG. 13: In FIG. 13 the immortalized human keratinocytes (N/TERTs) were differentiated for 7-10 days by removing the serum from the culture medium. As sign for differentiation the Cytokeratin 10 expression increased steadily over time in these cells. FIG. 13a shows the regulation of Cytokeratin 10 mRNA by real time PCR analysis in DOR overexpressing N/TERT cells compared to the control cells without DOR overexpression. FIG. 13b shows this downregulation of cytokeratin 10 expression in DOR overexpressing N/TERT cells (compared to mock cells without DOR overexpression) on protein level by specific western blot analysis of Cytokeratin 10. These results indicate the crucial role of the DOR system in keratinocyte differentiation (compare also the Cytokeratin 10 overexpression in epidermis of DOR knockout mice in Bigliardi et al, Differentiation, 2006).

FIG. 14: shows real time PCR analysis of POU2F3 mRNA. POU2F3 is the key transcription factor regulating K10 and sensory function of keratinocyte: until day 3 POU2F3 is down-regulated to promote cell proliferation function and later it is up-regulated to promote nerve regeneration and sensory restoration.

FIG. 15 depicts the significant reduction of proliferation in N/TERT keratinocytes after overexpression of the DOR receptor using an IncuCyte® Device and software (http://www.essenbioscience.com) to monitor cell proliferation. Data is shown after analysis by the Wound Confluence v 1.5 algorithm. The light blue line labelled “DOR ctrl” shows DOR over-expressing cells without exposure to ligand and the dark blue line labelled “DOR SNC” shows DOR over-expressing cells with exposure to specific DOR ligand SNC-80. The brown line labelled “GFP ctrl” shows proliferation in GFP control cells and the purple line labelled “GFP SNC” shows GFP control cells after exposure to SNC-80.

FIG. 16: FIG. 16 shows the measurement of effects of various DOR agonists and antagonists on wound closure/migration of primary human cultured fibroblasts in an in-vitro wound scratch model. FIG. 16a: SNC80 does not seem to change migration of human cultured Fibroblasts. However, Naltrindole (Nal) shows same significant improvement of wound closure/migration as the known stimulator TGFβ1. The graph shows the gap closure after 24 h (value 1=100% closure).

FIG. 16b shows the scratch wound closure after 24 h comparing various DOR and MOR antagonists by depicting the percentage of remaining gap after 24 h compared to time 0 of each experiment (time 0=100% gap). All opioid antagonists were used in a concentration of 100 nM. Naltriben is a specific DOR₂ antagonist, Naltrindole a mixed DOR_{1/2} antagonist, BNTX (7-Benzylidenenaltrexone) is a selective DOR₁ antagonist, beta-Funaltrexamine is an irreversible blocker of MOR (less KOR and even less DOR) and Cyprodime is a selective MOR antagonist (no DOR activity).

FIG. 17 shows the phenotype of organotypic 3D cultures using human immortalized keratinocytes (N/TERT) with overexpression of DOR, GFP control and wild type. The DOR overexpression results in a massive atrophy of epidermis with a reduced differentiation of the epidermis and missing corneal layer comparing to control cells (GFP control) and wildtype N/TERT cells. In other words, the DOR overexpression results in massive atrophy of Epidermis and the cells in Epidermis do not show differentiation into a corneal layer, unlike mock control cells (GFP ctrl.) and Wildtype (WT) N/TERT cells which show a multi-layered Epidermis with corneal layer.

FIG. 18 shows relevant results of a microarray assay performed with the keratinocytes cell line HeCat with overexpression of DOR (Lentivirus) after 48 h exposure to the DOR agonist SNC80. These results show clearly that the keratinocyte cell lines express taste (TAS2R14 and less TAS2R10) and various olfactory receptors. The microarray measures mRNA expression in these cells under various conditions.

The gene expression analysis was done as follows. 90% confluent HaCaT cells were aspirated and RNA was extracted with TRIZOL. RNA was further processed by the Lausanne Genomic Technologies Facility to prepare the probes for the Affymetrix whole transcriptome microarray analysis. The Ambion® WT Expression Kit was used. to generate sense strand cDNA from total RNA for further fragmentation and labelling using the Affymetrix GeneChip® WT Terminal Labeling Kit according to the manufacturer's recommendations. The processed samples were hybridized on a human gene 1.0 ST array and the generated expression profile analysed. Three replicates for each condition (DOR-overexpression vehicle treated, DOR-overexpression agonist treated, GFP control cells vehicle treated, and GFP control cells agonist treated) were scanned, normalized and data transformed to logarithmic scale to the base 2 for statistical calculations in a 2×2 factorial design.

FIG. 19 shows the expression of TAS2R14 in normal human skin (19a) and cultured human primary keratinocytes (19b). The taste receptor (green) is more expressed in the spinous and granular layer of epidermis (19a, left panel) and the negative control sections show no unspecific green staining (19a, right panel). The blue stain is a nuclear stain. The cultured human keratinocytes expressed TAS2R14 immunostaining in undifferentiated state (FIG. 19b, left panel) and 7 days post-differentiation with 1.2 mM calcium (FIG. 19b, right panel).

FIG. 20 shows the Calcium imaging of calcium responses of human primary keratinocytes and human skin cell lines (N/TERTs) to the specific TAS2R14 ligand Thujone 3 mM (FIG. 20a) or Flufenamic Acid FFA 3 mM (FIG. 20b).

FIG. 21 shows the concentration/response relationship in calcium responses of both human primary keratinocytes (FIG. 21a) and human skin cell lines nTERTs (FIG. 21b) to the specific TAS2R14 ligand Thujone 3 mM (FIG. 20a) or Flufenamic Acid FFA 3 mM (FIG. 20b). There seems to be for both ligands an all or nothing relationship that makes a cut-off in the range of 1-3 mM of ligand. To gain an idea of calcium movements during the agonist responses we used a spinning disc confocal to take time lapse Z-stacks (FIG. 21c) under different conditions. The upper panel shows a control condition with diluent added only, the middle panel shows the calcium distribution after exposure to 1 mM Thujone and the low panel shows the calcium distribution after exposure to 1 uM Ionomycine.

FIG. 22 a-e indicates the signal transduction pathway of the TAS2R14 receptor in human skin cells after blocking the various pathways with specific blockers. Suramin, a compound that uncouples G-proteins attenuates significantly Calcium responses by Thujone and FFA (FIG. 22a). This indicates that the Calcium signal is indeed G-protein related. The Calcium ATPase inhibitor Cyclopiazonic acid affected the calcium responsiveness to Thuj one or FFA (FIG. 22b), indicating that the intracellular (and not extracellular) calcium stores from Endoplasmatic reticulum are recruited by this specific response. Xestospongin blocks one of the possible G-protein related calcium release from ER and it is a specific IP3 receptor inhibitor. FIG. 22c shows a highly significant inhibition of the Thujone response in keratinocytes, suggesting that the IP3 mechanism is involved. Another possible pathway for the specific taste receptor signalling is a calcium release from ER by Ryanodine receptors. Ryanodine (FIG. 22d) and Dantrolene (FIG. 22e), both specific inhibitors of this pathway, significantly attenuate the response to Thuj one.

### DETAILED DESCRIPTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting. Therefore, though the following discussion relates to the cornified epithelia of skin, the methods, use and/or agents of the present invention can be transferred to all other types of epithelia of the external barrier and its appendages.

### DEFINITIONS

Unless defined otherwise, all technical and scientific terms used herein have the same meaning as is commonly understood by one of skill in art to which the subject matter herein belongs. As used herein, the following definitions are supplied in order to facilitate the understanding of the present invention. In the case of conflict, the present specification, including definitions, will control.

The term “comprise” is generally used in the sense of include, that is to say permitting the presence of one or more features or components. For completeness, it is noted that the term “comprise” encompasses the terms “consists essentially of” and “consists of”, which are used to refer to an exclusive list and that these terms can be used to replace “comprise” in all aspects and embodiments of this invention.

As used in the specification and claims, the singular form “a”, “an” and “the” include plural references unless the context clearly dictates otherwise.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects (age 0 to 130) whether male or female are intended to be covered. This is true for humans and companion animals, for example horses, cats and dogs.

The term “selective antagonists of delta-opioid receptors” or “selective delta-opioid receptor antagonists” as used herein denotes any compound which has a higher binding affinity for the delta-opioid receptor than for any other opioid receptor. In particular the term denotes antagonist compounds capable of ligand-receptor binding to at least one opioid receptor, the antagonist compound having a higher binding affinity for the delta-opioid receptor than for any other receptor normally found in the vicinity of the delta-opioid receptor in the human or animal body. Similarly, a “selective DOR agonist” refers to agonist compounds capable of ligand-receptor binding to at least one opioid receptor, the agonist compound having a higher binding affinity for the delta-opioid receptor than for any other receptor normally found in the vicinity of the delta-opioid receptor in the human or animal body.

The term “agonist” as used herein refers to a drug which binds to a receptor and activates it, producing a pharmacological response (contraction, relaxation, secretion, enzyme activation, etc.).

The term “antagonist” as used herein refers to a drug which attenuates the effect of an agonist. It may be competitive (or surmountable), i.e. it binds reversibly to a region of the receptor in common with an agonist, but occupies the site without activating the effector mechanism. The effects of a competitive antagonist may be overcome by increasing the concentration of agonist, thereby shifting the equilibrium and increasing the proportion of receptors which the agonist occupies. However, it is now known that certain antagonists can affect Receptor trafficking and therefore improve agonist actions indirectly.

At present, there is some uncertainty as to the specific types of DOR receptors which may exist, e.g. monomeric, heteromeric, homomeric, DOR1, DOR2. The specific receptor families of DOR1 and DOR2 have not been fully established as yet, but the DOR1 family has been shown to include at least a DOR-KOR heterodimer, and the DOR2 family includes at least a DOR-MOR heterodimer. Further, it has been shown that a heterodimer ligand may be a combination of linked or unlinked ligands for the monomers, or a single ligand allosterically binding to the heterodimer.

The term “an effective amount” refers to an amount necessary to obtain a physiological effect. The physiological effect may be achieved by one application dose or by repeated applications. The dosage administered may, of course, vary depending upon known factors, such as the physiological characteristics of the particular composition; the age, health and weight of the subject; the nature and extent of the symptoms; the kind of concurrent treatment; the frequency of treatment; and the effect desired and can be adjusted by a person skilled in the art.

Alternatively, the term “antagonists of opioid receptors” as used herein may denote any compound which inhibits the opioid receptor signalling or down-regulates the expression of the opioid receptors in human skin cells in particular melanocytes but not limited to them. The opioid receptor antagonists may be opioid analogues, e.g., (CAS number given in parenthesis where appropriate) Naloxone (465-65-6); Naloxonazine (82824-01-9); Cyprodime (118111-54-9); β-Funaltrexamine (72782-05-9); Nalbuphine (20594-83-6); RX 8008M (40994-80-7); SDZ 210-096 (109026-86-0); Clocinnamox (117332-69-1); NIH 10236 (88167-37-7); BU 165 (173321-27-2); BU 164 (173429-52-2); BU 158 (173429-53-3); BU 160 (173429-56-6); BU 161 (173429-57-7); BU 162 (173429-58-8); Buprenorphine (52485-79-7); IOXY (141392-28-1); NPC 168 (115160-07-1); Naloxazon (73674-85-8); N-Methylnaloxonium Iodide (93302-47-7); 3-Methoxynaltrexone Hydrochloride; 7-Benzylidenenaltrexone 129468-28-6); Naltrindole Isothiocyanate (126876-64-0); BNTX (153611-34-8); Naltriben (111555-58-9); Naltrexone (16590-41-3); Nalmefene (55096-26-9); β-Chlornaltrexamine (67025-94-9); Diprenorphine (14357-78-9); nor-Binaltorphimine (105618-27-7); Naltrindole (111555-53-4); or (poly)peptides, e.g., CTAP (103429-32-9); TCTOP (115981-70-9); TCTAP (115981-71-0); CTOP (103429-31-8); Tyr MIF-1 (77133-61-0); CCK-8 (25126-32-3); CG 3703 (90243-66-6); compounds disclosed in Peptide Research 1995, 8(3), 124-37, Proceedings of the National Academy of Sciences of the United States of America (1993), 90(22), 10811-15, Regulatory Peptides (1994), (Suppl. 1), S53-S54; SMS 201-995 (83150-76-9); e-PMTC as disclosed in Medicinal Chemistry Research (1994), 4(4), 245-53; CTP (103335-28-0); TIPP (146369-65-5); ICI 154129 (83420-94-4); ICI 174864 (89352-67-0); or piperidine derivatives, e.g., the compounds disclosed in J. Med. Chem. 1993, 36(20); 2833-41, EP 657428 and EP 506478; or may belong to different structures, such as Quadazocine (71276-43-2); Flumazenil (78755-81-4); BIT (85951-65-1); Dezocine (53648-55-8); Ciramadol (63269-31-8). Ginseng root extract like in Journal of Ethnopharmacology (1994), 42(1), 45-51; Rimcazole (75859-04-0); MR 2266 (56649-76-4); and WIN 44441-3 (71276-44-3).

Further opioid receptor antagonists, particularly the μ-receptor, and assays for determining their efficacy in binding to the receptors have been disclosed in e.g., WO 02/098422, U.S. Pat. No. 5,270,328, US 2001/0036951 A, WO 01/42207, WO 01/37785, WO 01/41705, WO 03/101963, WO 2004/005294, WO 2004/014310, WO 2004/038005, and WO 2004/051264.

Alternative μ-opioid receptor antagonists for use according to the invention include, but are not limited to, Naloxone; Naloxonazine; piperidine derivatives such as quoted above; Cyprodime; β-Funaltrexamine, Nalbuphine, CTAP, TCTOP, TCTAP, CTOP, Quadazocine, Flumazenil, RX 8008M, SDZ 210-096, Tyr MIF-1, CCK-8, CG 3703, Clocinnamox, peptides such as disclosed in Peptide Research 1995, 8(3), 124-37, Proceedings of the National Academy of Sciences of the United States of America (1993), 90(22), 10811-15, Regulatory Peptides (1994), (Suppl. 1), S53-S54; NIH 10236, BU 165, BU 164, BU 158, BU 160, BU 161, BU 162, Buprenorphine, IOXY, SMS 201-995, e-PMTC as disclosed in Medicinal Chemistry Research (1994), 4(4), 245-53; CTP, BIT, NPC 168, Naloxazon, Dezocine and Ciramadol. Other, typical κ-, δ-receptor or non-selective (still binding to μ-receptors as well) antagonists for use herein include, but are not limited to: N-Methylnaloxonium Iodide, 3-Methoxynaltrexone Hydrochloride; 7-Benzylidenenaltrexone, Ginseng root extract as disclosed in Journal of Ethnopharmacology (1994), 42(1), 45-51; Rimcazole, Naltrindole Isothiocyanate, BNTX, TIPP, Naltriben, Naltrexone, ICI 154129, MR 2266, WIN 44441-3, Nalmefene, β-Chlornaltrexamine, ICI 174864, Diprenorphine, nor-Binaltorphimine and Naltrindole.

Further opioid receptor antagonists which can be used for the purposes of the present invention comprise Epigallocatechin 3,5-Digallate (37484-73-4), Irigenol Hexaacetate (103652-04-6), Irigenol ex Iris spp (4935-93-7), Berbamine Hydrochloride (5956-76-3), Quercetagetin (90-18-6), Acetylshikonin (24502-78-1), 2′,3′,4′,3,4-Pentahydroxychalcone (484-76-4), beta,beta-Dimethylacryl shikonin (24502-79-2), 2,3-Dimethoxy-5-methyl-1,4-benzoquinone (605-94-7), 2,3-Dimethoxy-5-methylhydroquinone (3066-90-8), 2,3-Dimethoxy-1,4-benzoquinone (3117-02-0), 2,3-Dimethoxyhydroquinone (52643-52-4), Delphinidin chloride (528-53-0), Aureusidin (38216-54-5), Isocembrol (25269-17-4) and Robinetin (490-31-3) without being limited thereto.

The above-identified opioid receptor antagonists or their salts may be used as racemates or as pure enantiomers, or diastereomers or mixtures thereof. Preferably, pure enantiomers are used. If one or more chiral centers are present the optical purity of the mixture is preferably ≧80% ee, more preferably ≧90% ee, most preferably ≧95% de. If two or more chiral centers are present the purity of the mixture is preferably ≧80% de, more preferably ≧90% de, most preferably ≧95% de.

In all embodiments of the invention the term ‘opioid receptor antagonists’ also encompasses any material or extract of a plant containing at least one opioid receptor antagonist of in an amount of at least 30 weight-% (i.e. from 30 to 100 weight-%), preferably in an amount of at least 50 weight-% (i.e. from 50 to 100 weight-%), more preferably in an amount of at least 70 weight-% (i.e. from 70 to 100 weight-%), most preferably in an amount of at least 90 weight-% (i.e. from 90 to 100 weight-%), based on the total weight of the plant material or extract. The terms “material of a plant” and “plant material” used in the context of the present invention mean any part of a plant.

Further, derivatives of these compounds as appropriate, such as esters, amides, nitriles, oximes, imines, hydrazones, ethers, acetals, semiacetals may also find use. The ester or ether groups may for example be derived from straight or branched alkyl groups having 1 to 26 carbon atoms or from substituted or unsubstituted straight or branched aliphatic, araliphatic or aromatic carboxylic acids having 1 to 26 carbon atoms. Examples of etherified hydroxy groups further include glycoside groups. Examples of esterified hydroxy group further include glucuronide or sulfate groups.

The term “skin sensation” conditions denotes distinct conscious perception caused by physiological or non-physiological stimulation on skin which can be pleasant sensations (sunlight, warmth, soft) and feeling euphoria, well-being, elation, happiness, excitement, and joy or unpleasant sensation such as pain, itch, tactile, burning, tickling, tingling, prickling, stinging, stretching, swelling and foreign body sensations. It can act as warning signals, as well as forming parts of social and physiological interactions between individuals and their environment. For example, the stroking 4 cm/second cause pleasant sensation. Foreign body sensations may include formication. Skin sensation conditions may also include sensitive skin.

As used herein, the term “topical pharmaceutical composition” refers to a composition which is applied to body surfaces such as appendages, or more particularly the skin or mucous membranes, and which has a topical effect, i.e. local effect contrasting with systemic effects. Topical pharmaceutical compositions are designed for topical administration. The composition includes also all different types of carriers or vehicles used in formulations for topical application.

The phrase “pharmaceutically acceptable salt(s),” as used herein includes, but is not limited to, salts of acidic or basic groups that may be present in the compounds of the invention. Compounds of the invention that are basic in nature are capable of forming a wide variety of salts with various inorganic and organic acids. The acids that may be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide, and pamoate (i.e., 1,1′-methylene-bis-(2-hydroxy-3-naphthoate)).

The term “solvate” refers to solvates that are formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

As used herein, the terms “treat”, “treating” and “treatment”, and the like refer to reversing, healing, alleviating, or inhibiting the progress of the disorder or condition to which such term applies, or one or more symptoms of such disorder or condition. The term “treatment”, as used herein, refers to the act of treating, as “treating” is defined immediately above. The term “treatment” or “treating” includes the reduction in appearance of skin imperfections irrelevant of the mechanism of action. One of ordinary skill in the art will appreciate that the endpoint of treatment chosen in a particular case will vary according to the disease, condition, or disorder being treated, the outcome desired by the patient, subject, or treating physician, and other factors. For topical compositions, the endpoint can be determined by the patient's, or the treating physician's, satisfaction with the results of the treatment. Alternatively, endpoints can be defined objectively.

### Skin Wounds, Aging Conditions and Sensation Conditions

In light of studies suggesting the importance of DOR in cell differentiation, migration, adhesion, cytokeratin and cytokine expression and regulation of metalloproteinases and connective tissue in skin (Bigliardi et al, Differentiation, 2006), the activation of DOR is expected to improve wound healing and to generally promote healthy epithelia. Selective AGONISTS for the DOR are the most promising therapeutic candidate as they are known to stimulate the activity of these receptors without the adverse side effects associated with morphine and other opioid drugs, e.g. as an analgesic.

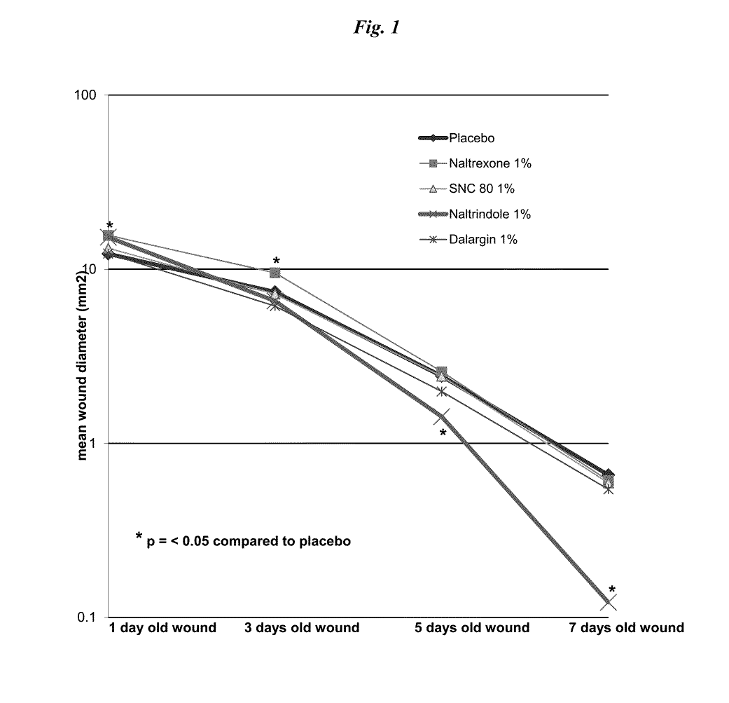

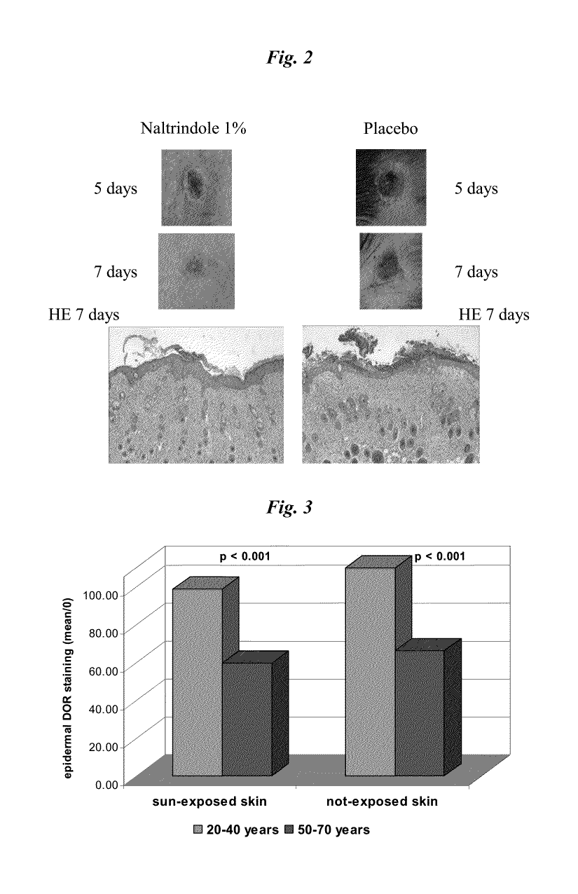

To the contrary to what was believed that DOR agonists would improve wound healing, surprisingly the Applicants have observed that a selective DOR antagonist, such as Naltrindole, improved the wound healing by 3 days (FIGS. 1 and 2). In particular, the Applicants have surprisingly observed that a selective DOR antagonist, such as Naltrindole, significantly improved wound healing—in the case of 6 mm punch wounds in mice, by 3 days compared to a placebo. Naltrindole is a highly potent, highly selective DOR antagonist, which binds almost exclusively to the DORs (Portoghese P S, Sultana M, Takemori A E. “Naltrindole, a highly selective and potent non-peptide delta opioid receptor antagonist.” European Journal of Pharmacology. 1988 Jan. 27; 146(1):185-6). While it is known to selectively and effectively block the action of DORs, the present disclosure shows for the first time its effectiveness to promote wound healing.

The present invention discloses the first time that the selective DOR antagonist and/or the selective ligand for sensory receptor may have effects on epithelial cells, fibroblasts and melanocytes. Thus, according to one aspect of the invention there are provided:

(i) a selective DOR antagonist and/or a selective ligand for sensory receptor for use in the treatment of epithelial or more particularly dermal conditions, such as skin wounds, skin aging conditions and/or skin sensation conditions;
(ii) the use of a selective DOR antagonist and/or a selective ligand for sensory receptor for the manufacture of a medicament for treating epithelial conditions, such as skin wounds, skin aging conditions and/or skin sensation conditions; or
(iii) a method for treating epithelial conditions, such as skin wounds, skin aging conditions and/or skin sensation conditions by administering an effective amount of a selective DOR antagonist, a combination of selective DOR antagonist and agonist and/or a selective ligand for sensory receptor to a subject in need thereof.

Skin wound, as used herein, is defined as a breach in the continuity of skin tissue (e.g. epidermal and dermal), which is caused by direct injury to the skin. For example, skin wound can be caused by burn, chemical and/or mechanical injury to the skin. Skin wounds that may be treated by the method of the present invention comprises but not limited to punctures, incisions including those produced by a variety of surgical procedures, excisions, lacerations, abrasions, atrophic skin or necrotic wounds and burns including large burn areas, sun burns and UV burns.

Treating skin wounds according to the present invention includes promoting dermal and epidermal wound healing, minimizing scarring decreasing dysaesthesia in scars (itching, pain, hypersensibility, hyposensibility) by helping reinnervation, improving the aesthetics of scars (post operative, burns, postinflammatory), facilitating and enhancing healing in burned and/or traumatized tissues, and reducing stretch marks.

Aging skin loses some of its protective fatty layer and becomes more fragile. Also aging skin decreases in the ability to produce protein fibers and substrate mass and thus become thinner, leading to a reduction in skin elasticity and the formation of wrinkles. Skin atrophy is another natural result of aging. Skin atrophy happens when the layer of skin called the dermis and/or epidermis decreases in thickness and size. The dermis is the portion of the skin that produces collagen which gives skin its structure and suppleness. The dermis is also the portion of the skin that produces extracellular matrix (ECM) proteins which also give skin structure and suppleness. Without the benefit of collagen or optimal ECM composition, the skin becomes thin, wrinkled, and prone to bleeding and bruising. The outermost layer of the skin called the epidermis also thins out when the skin becomes atrophic.

The dermal fibroblasts and epidermal cells together play a crucial role in the homeostasis of the entire skin, which is important in skin ageing and wound healing. We have observed in human skin (FIG. 3) that the DOR expression in Epidermis is reduced in aged skin (compared to young skin) and this is independent from photoageing. Therefore, DOR is a marker and regulator of biological ageing in skin (and NOT photoageing).

Fibroblasts and epidermal cells are crucial components of the hair follicle and the DOR has been found previously in skin appendages, such as hair follicles (Bigliardi et al, Exp Dermatol, 2009), nails and sebaceous/sweat glands and therefore the same treatment effects of DOR antagonists and/or combination of DOR antagonists and agonists can be expected in disorders of hair, nails and sebaceous glands.

In addition, the results disclosed herein show that in-vitro melanocytes are affected by DOR system (FIG. 7) and clinical studies (FIG. 4) have shown that DOR system is correlated with the appearance of age-induced lentigenes (hyperpigmentations) in humans. Therefore, it is expected that specific DOR antagonists and the combination of DOR antagonists and agonists can be used to treat pigmentary disorders (hyper-, hypo- and depigmenations).

Treating skin aging conditions according to the present invention includes methods of ameliorating and treating skin aging conditions selected from the group comprising wrinkles, skin discoloration, rosacea, senile angiomas, vessel fragility with haematomas, photoaging, lentigines, loss of elasticity, increased fragility of skin, dry and itchy skin, delayed wound healing and also disorders of skin appendages (hair, nail, sebaceous and sweat glands) and pigmentation. More in particular, treating skin aging conditions according to the present invention includes methods of ameliorating and treating skin aging conditions selected from the group comprising wrinkles, skin discoloration, rosacea, senile angiomas, vessel fragility with haematomas, photoaging, lentigines, loss of elasticity, increased fragility of skin, dry and itchy skin and delayed wound healing.

In one embodiment, the selective DOR antagonist and/or the selective ligand for sensory receptor is administered topically to the appropriate skin area of a subject in need of such treatment in an amount effective to stimulate DOR in cells, thereby promoting wound healing and ameliorating skin aging conditions.

Said topical administration can also include, in combination with said selective DOR antagonist, one or more DOR agonist. Preferably the DOR agonist may be selected from the group comprising SNC-80, BW373U86, DPI-287, or DPI-3290. Further, said topical administration can also include, in combination with said selective DOR antagonist, one or more pharmaceutically active ingredient selected from the group consisting of an antibacterial agent, an antiviral agent, an antifungal agent, an antiparasitic agent, an antiinflammatory agent, an analgesic agent and an antipruritic agent. Alternatively, said topical administration can also include, in combination with said selective DOR antagonist, one or more DOR or MOR or taste receptor or olfactory receptor agonist simultaneously or sequentially. Preferably the DOR agonist is selected from the group comprising SNC-80, BW373U86, DPI-287, or DPI-3290, for example. MOR agonist may be selected from the group comprising morphine, dermorphine, endomorphine, fentanyl, codein, for example.

### Delta Opioid Receptor (DOR) Antagonist

It has been reported that MOR and DOR antagonists decrease proliferation and increase neurogenesis in cultures of rat adult hippocampal progenitors and regulate proliferation in both adult and embryonic neural tissues in vivo (Persson A I et al., Eur. J. Eur. Neurosci. 17: 1159-1172 (2003)). Similarly DOR antagonists in skin reduce MAPK signalling and may decrease the levels of c-fos, c-jun and c-junD, involved in formation of AP-1 complexes that mediate less transcription followed by less translation of genes such as PCNA, CDK2 and CDK4. The inhibition of the cell proliferation by DOR antagonist indicates that opioid receptor ligands participate in cell cycle, differentiation and cell lineage decision processes. Without being bound by theory, it is thought that skin cells take on differentiation pathway and promote neurogenesis, which depends on the local environment in vivo rather than on intrinsic properties of the cells. This neurogenesis is probably mainly responsible for the improved wound healing we observed. Similarly, the use of the selective ligands for sensory receptor of the present invention in anti-inflammatory and wound healing promotion therapy can also be well explained through this mechanism.

The Applicants revealed a very significant advantage of a specific DOR antagonist over the DOR agonist SNC 80 and the enkephalin analogue Dalargin. Naltrexone, an antagonist of δ-, κ-, and μ-opioid receptors was also not effective. It seems that the specific and potent DOR agonist SNC 80 induced downregulation of opioid receptor expression by internalization and degradation and that the specific DOR antagonist upregulated DOR. There is probably more than enough endogenous ligand (enkephalins, endorphins) in the wounded skin, so that the upregulation of DOR by the antagonist resulted in an enhanced wound healing response.

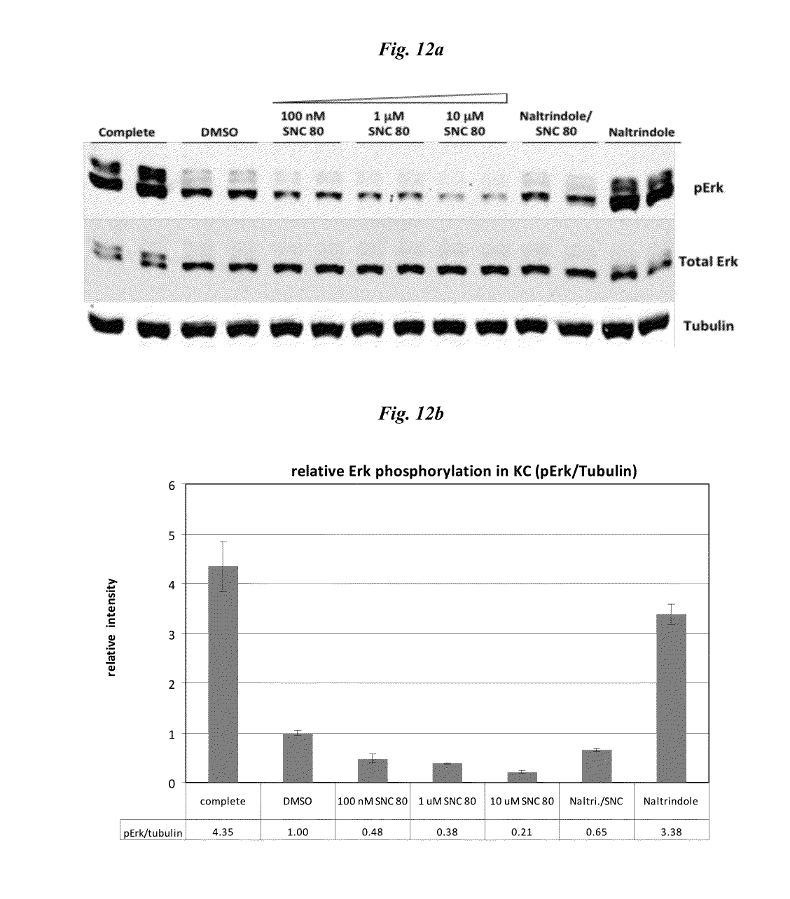

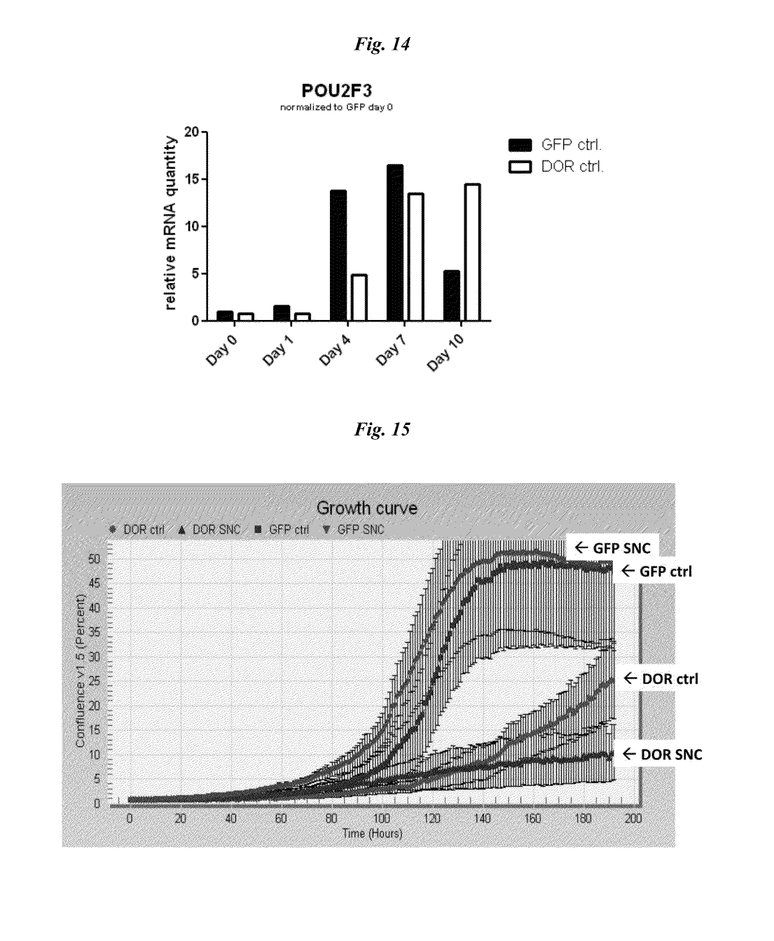

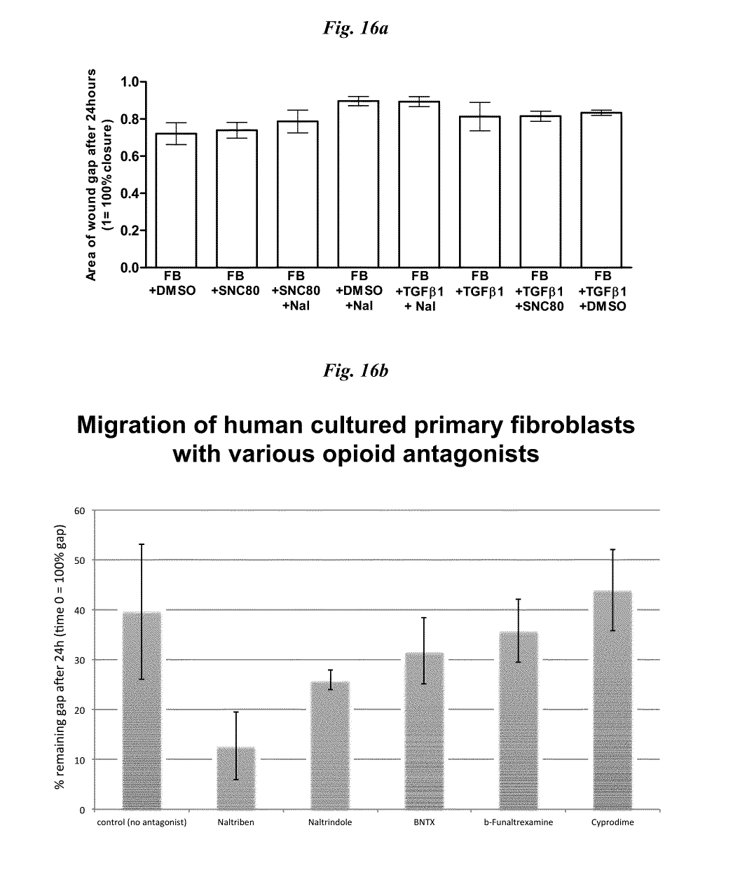

Selective DOR antagonist Naltrindole has shown strong stimulating effect in primary human keratinocytes and fibroblasts via signaling pathway of MAPK pathway ERK1/2 (FIGS. 12a and 12b) and POU2F3 (FIG. 14). Activated ERK1/2 is able to interact with and phosphorylate a large number of target proteins, including cytosolic and membrane proteins (e.g. PLA2, Syk), nuclear (Elk-1, c-fos. c-jun, c-myc and Stat3) and cytoskeletal proteins (neurofilament and paxillin). Expression of different genes can be regulated in the consequence. ERK can influence cellular processes like differentiation or proliferation, processes involved in skin homeostasis and wound healing. This is the first time to show that DOR antagonists Naltrindole on molecular mechanism level its great activity and explains its wound healing stimulating effect. In skin the dermal fibroblasts play a crucial role in ageing processes (e.g. wrinkling) and also in wound healing by providing the extracellular matrix. The experiments (FIG. 16) have clearly shown that the DOR system is functionally expressed in human fibroblasts and that the DOR antagonists, in particular antagonists with DOR₂ effects, increases the mobility of these Fibroblasts. This underlines the biological and functional significance of the opioid receptor system throughout the entire skin.

In addition, POU2F3 pathway determine expression and function of bitter taste receptor in the epithelial cells and differentiate different stimuli of sweet, umami and bitter taste. Its activation can play an important role in skin defense, repair and regulation of sensation.

The Applicants have also shown that epidermal expression of DOR is significantly reduced in aging process and that there is a negative correlation between DOR and development of lentigenes (sun-spots) and wrinkles. The Applicants have also shown that the up-regulation of DOR mRNA, and hence expression of DOR in human skin cells, can be done by a selective DOR antagonist, such as Naltrindole (FIG. 12b). Normal DOR expression is an indication for healthy, youthful skin (less wrinkles and less age spots).

The Applicant's results show that there really exists a regulation of DOR expression in different human skin cells. However, this regulation depends from the cell type and the exposure time and the concentration of the ligands. The in-vitro assay data show that selective delta-opioid antagonists can upregulate the DOR receptor system in skin. The natural ligand enkephalin, released in wounds by keratinocytes and immune cells, can contribute to this regulation. Thus it is possible either to further add an agonist or rely on endogenous agonists, already supplied by the skin and immune cells, in the treatment methods.

The selective DOR antagonist of the present invention is selected from the group comprising benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

Preferably the selective DOR antagonist of the present invention is selected from the group comprising naltrindole, methylnaltrindole, N,N(Me)₂-Dmt-Tic-OH (N,N(Me)₂-Dimethyl-tyrosine-1,2,3,4-tetrahydroisoquinoline-3-carboxylic acid-OH), SoRI-9409 (5′-(4-Chlorophenyl)-17-(cyclopropylmethyl)-6,7-didehydro-3,14-dihydroxy-4,5-α-epoxypyrido-[2′,3′:6,7]morphinan), naltriben or pharmaceutically acceptable salts thereof. Most preferably the selective DOR antagonist is naltrindole, naltriben or pharmaceutically acceptable salts and/or solvates thereof.

In some embodiments, the selective DOR antagonist may be a DOR₂ antagonist or a pharmaceutically acceptable salt and/or solvate thereof. Preferably, the selective DOR₂ antagonist is naltriben, TIPPψ (H-Tyr-TicPsi[CH(2)NH]Phe-Phe-OH) or a pharmaceutically acceptable salt and/or solvate thereof. In alternative embodiments, the selective DOR antagonist may be a DOR₁ antagonist or a pharmaceutically acceptable salt and/or solvate thereof. Preferably, the selective DOR₁ antagonist is benzylidenenaltrexone or a pharmaceutically acceptable salt and/or solvate thereof.

Some progress has been made in the development of highly selective opioid antagonists. For example, Portoghese et al. (U.S. Pat. No. 4,816,586) disclose certain opiate analogs which possess high selectivity and potency for blocking delta receptors, e.g. in the treatment of shock. Minimal involvement was observed at mu and kappa opioid receptors. One of the highly selective analogs disclosed in U.S. Pat. No. 4,816,586 has been named “naltrindole” or “NTI” (See P. S. Portoghese et al., J. Med. Chem., 31, 281 (1988)).

The selective DOR antagonist can be also any compound selected with (a) the binding assay and/or (b) MAP kinase activation assay.

(a) Naltrindole and other selective DOR antagonist, as well as the ligands selective for delta (δ) opioid receptors, such as pCI-DPDPE and Deltorphin II inhibits [³H]naltrindole binding with nanomolar IC₅₀ values. Ligands selective for mu (μ) and kappa (κ) opioid receptors are only effective inhibiting [³H]naltrindole binding at micromolar concentrations.
(b) Naltrindole and other selective DOR antagonist can promote phosphorylation of MAP kinase such as Erk at less than or equal to 10 μM but not other non-selective antagonists at the same concentration

The above-identified DOR antagonists can suitably be used in the form of pharmaceutically acceptable salts, with inorganic acids, e.g. hydrochlorides, hydrobromides, sulfates, phosphates, or organic acids, e.g. methanesulfonates, p-toluenesulfonates, carbonates, formats, acetates, oxalates, lactates; or as hydrates as appropriate. The above-identified DOR antagonists or their salts may be used as racemates or as pure enantiomers, or diastereomers or mixtures thereof. Preferably, pure enantiomers are used.

In all embodiments of the invention the term “delta opioid receptor antagonists” or “DOR antagonists” also encompasses any material or extract of a plant containing at least one opioid receptor antagonist of in an amount of at least 30 weight-% (i.e. from 30 to 100 weight-%), preferably in an amount of at least 50 weight-% (i.e. from 50 to 100 weight-%), more preferably in an amount of at least 70 weight-% (i.e. from 70 to 100 weight-%), most preferably in an amount of at least 90 weight-% (i.e. from 90 to 100 weight-%), based on the total weight of the plant material or extract. The terms “material of a plant” and “plant material” used in the context of the present invention mean any part of a plant.

Further, derivatives of these compounds as appropriate, such as esters, amides, nitriles, oximes, imines, hydrazones, ethers, acetals, semiacetals may also find use. The ester or ether groups may for example be derived from straight or branched alkyl groups having 1 to 26 carbon atoms or from substituted or unsubstituted straight or branched aliphatic, araliphatic or aromatic carboxylic acids having 1 to 26 carbon atoms. Examples of etherified hydroxy groups further include glycoside groups. Examples of esterified hydroxy group further include glucuronide or sulfate groups.

### Delta Opioid Receptor (DOR) or Mu Opiod Receptor Agonists

As noted herein, aspects and embodiments of the invention, may make use of an opioid receptor agonist in combination with the DOR antagonist. Preferably, the opioid receptor agonist is a mu opioid receptor (MOR) agonist, a DOR agonist or a pharmaceutically acceptable salt and/or solvate thereof. For example, the opioid receptor agonist may be selected from the group consisting of SNC-80, BW373U86, DPI-287 and DPI-3290 and Met-enkephalin, (D-Pen²,D-Pen⁵)enkephalin, (D-Ala²)deltorphin II, 7-spiroindanyloxymorphone, ADL-5859, BU-48, DADLE, deltorphin, D-Pen², D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

In certain embodiments, the opioid receptor agonist is preferably a selective DOR agonist. For example, the DOR agonist may be selected from the group consisting of SNC-80, BW373U86, DPI-287 and DPI-3290 or pharmaceutically acceptable salts and/or solvates thereof.

In other aspects and embodiments, the combination of a selective DOR antagonist with an opioid receptor agonist may refer to a single compound, such as a mu-delta agonist-antagonist (MDAN) compound or pharmaceutically acceptable salts and/or solvates thereof. For example, the MDAN compound may comprise a mu opioid receptor (MOR) agonist linked to a DOR antagonist by a linker comprising a backbone of at least 16 atoms or pharmaceutically acceptable salts and/or solvates thereof. In certain embodiments, the MOR agonist linked to a DOR antagonist by a linker may be oxymorphone or a derivative thereof. For example, the mu-delta agonist-antagonist compound may have a general formula (I):

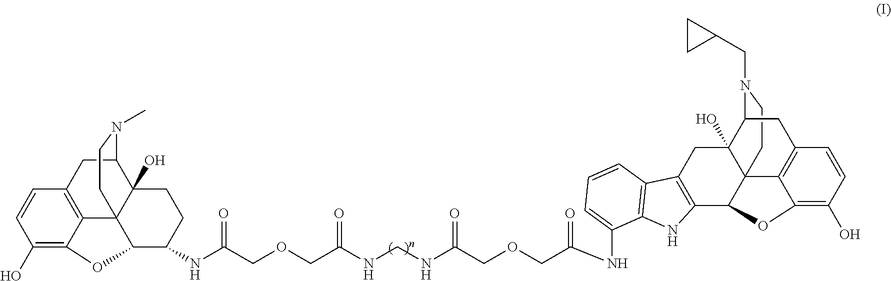

wherein n represents an integer of from 2 to 7.

### Taste Receptor Ligands and Olfactory Receptor Ligands

New data prove the presence of specific olfactory receptors (OR2T4, OR11G2) and even functionally active specific taste receptors (TSR2R14) in keratinocytes. These specific receptors to chemical stimuli are previously described in the olfactory and taste receptor, but never in skin cells.

The selective ligand for sensory receptor may be include but not limited to thujone and flufenamic acid.

Taste receptor ligands and olfactory receptor ligands (with all combination of concentration) can be used to improve wound healing, anti itching, sensing danger, sensitive and irritated skin and can be used in cosmetic and fragrant products for anti ageing, rejuvenation and sensitive skin. Or, more particularly, Taste receptor ligands and olfactory receptor ligands (with all combination of concentration) can be used to improve wound healing, anti-itching, treatment of tumors, sensing danger, sensitive and irritated epithelia and can be used in cosmetic and fragrant products for anti ageing, rejuvenation and sensitive skin. The skin cells act as receptor for different taste and olfactory signals and these signals are directly communicated via the peripheral nerve system to the CNS. This can finally show that olfactory and taste ligands have indeed effects on general well-being and well feeling. We can use skin cells (keratinocytes, fibroblast, melanocyte, dendritic cells and other skin immune cells) and co-culture experiments with peripheral sensory nerve fibres by overexpressing taste and olfactory receptors to screen the molecules, which will be used in above application. This was discovered in keratinocyte but could be likely in melanocyte and other epithelial cell types such as corneal epithelial cells. The cell system can also be used to develop for cosmetic industry a high throughput screening for olfactory and taste products, used mainly in fragrances and essential oils. The same concept can be applied to other peripheral epithelial tissues such as respiratory, gastro-intestinal and mucosal (oral, anal or uro-genital).

New data linking the DOR system in skin cells to the expression of chemical sensor receptors, taste and olfactory receptors, suggesting in addition that specific ligands for the taste receptor (TAS2R14) and the olfactory receptors (OR2T4, OR11G2) can be used to improve the skin sensation and wound repair. In addition to the specific agonists and antagonists of the DOR system, these taste and olfactory receptor ligands can also be used in skin by using above mentioned pharmacological applications alone or in combination for treatment of sensitive skin and unpleasant skin sensations.

### Topical Pharmaceutical Composition

In a further aspect, the present invention provides a topical pharmaceutical composition for treating epithelial conditions, such as skin wounds, skin aging conditions and/or skin sensation conditions, comprising at least one selective DOR antagonist and a pharmaceutically acceptable carrier. Optionally, the topical pharmaceutical composition may further comprise an opioid receptor agonist, as described herein. Alternatively, the topical pharmaceutical composition may comprise an MDAN compound, as described herein.

Preferably said selective DOR antagonist is selected from the group comprising naltrindole, methylnaltrindole, N,N(Me)₂-Dmt-Tic-OH (N,N(Me)₂-Dimethyl-tyrosine-1,2,3,4-tetrahydroisoquinoline-3-carboxylic acid-OH), SoRI-9409 (5′-(4-Chlorophenyl)-17-(cyclopropylmethyl)-6,7-didehydro-3,14-dihydroxy-4,5-α-epoxypyrido-[2′,3:6,7]morphinan), naltriben or pharmaceutically acceptable salts thereof. Most preferably said selective DOR antagonist is naltrindole or pharmaceutically acceptable salts thereof. Further alternatives (such as selective DOR₁ and DOR₂ antagonists are described herein).

The topical compositions according to the invention comprise the selective DOR antagonist in an amount of at least 0.00001 wt.-%. Preferably from about 0.00001 wt.-% to about 20 wt.-%, more preferably from about from about 0.0001 wt.-% to about 10 wt.-%, most preferably from about from about 0.1 wt.-% to about 1 wt.-%. The dosage depends from transcutaneous absorption, if effect in epidermis or dermis, indication and application time.

The topical pharmaceutical compositions of the present invention are preferably applied at least once per day, preferably twice or triple times a day, or several times a day. Alternatively the topical pharmaceutical compositions of the present invention may be applied at various frequencies, e.g. less than once a day or as frequently as necessary (e.g. more than three times a day), e.g. once in two days, once a week, or as frequently as needed for relief, e.g. more than three times a day. The appropriate dosage regime may be determined by clinical studies. The amount of the topical pharmaceutical composition, which is to be applied to the skin, depends on the concentration of the selective DOR antagonist and optionally other active ingredients in the compositions and the desired pharmaceutical effect. For example, this amount will be affected by the type of vehicle or carrier and topical formulation used with other active ingredients to get the desired cosmetic or pharmaceutical effect. For example, application can be such that a cream is applied to the skin. A cream is usually applied in an amount of 2 mg cream/cm² skin. The amount of the topical pharmaceutical composition which is applied to the skin is, however, not critical, and if with a certain amount of applied topical pharmaceutical composition the desired effect cannot be achieved, a higher concentration of the selective DOR antagonist can be used e.g. by applying more of the topical pharmaceutical composition or by applying topical pharmaceutical compositions which contain more selective DOR antagonist. The composition according to the invention can also contain one or more additional pharmaceutically active ingredient.

The topical pharmaceutical composition according to the present invention can also include one or more DOR or MOR agonist. The DOR agonist may be selected from the group comprising SNC-80, BW373U86, DPI-287, or DPI-3290, for example. MOR agonist may be selected from the group comprising morphine, dermorphine, endomorphine, fentanyl, codein, for example. In addition, the application of specific DOR antagonists with MOR or DOR agonists or taste or olfactory receptor ligands can be simultaneously in one single formulation or sequentially in various formulations. More in particular, the topical pharmaceutical composition according to the present invention can also include one or more DOR agonist. Preferably DOR agonist is selected from the group comprising SNC-80, BW373U86, DPI-287, or DPI-3290.

The topical pharmaceutical composition according to the present invention can also include one or more selective ligand for sensory receptor.

Again, the topical composition according to the invention may comprise the one or more DOR agonist and/or the one or more selective ligand for sensory receptor, in an amount of at least 0.00001 wt.-%. Preferably from about 0.00001 wt.-% to about 20 wt.-%, more preferably from about from about 0.0001 wt.-% to about 10 wt.-%, most preferably from about from about 0.1 wt.-% to about 1 wt.-%. The dosage depends from transcutaneous absorption, if effect in epidermis or dermis, indication and application time.

The topical compositions according to the invention may comprise the selective DOR antagonist together with the one or more DOR agonist and/or the one or more selective ligand for sensory receptor in the same composition, for simultaneous administration. Alternatively, the selective DOR antagonist, the one or more DOR agonist and/or the one or more selective ligand for sensory receptor may be in separate topical compositions, which may be the same pharmaceutical dosage form or different dosage forms. For example, the selective DOR antagonist may be provided as a cream, whereas the one or more DOR agonist and/or the one or more selective ligand for sensory receptor may be provided in a patch, or vice versa. This allows concomitant or sequential administration in different sequences, as appropriate. The various compositions may be included together with instructions as part of a kit. Alternatively, the topical compositions according to the invention may comprise the selective DOR antagonist together with the one or more opioid receptor agonist and/or the one or more selective ligand for sensory receptor in the same composition, for simultaneous administration. Alternatively, the selective DOR antagonist, the one or more opioid receptor agonist and/or the one or more selective ligand for sensory receptor may be in separate topical compositions, which may be the same pharmaceutical dosage form or different dosage forms. For example, the selective DOR antagonist may be provided as a cream, whereas the one or more opioid receptor agonist and/or the one or more selective ligand for sensory receptor may be provided in a patch, or vice versa.

The topical pharmaceutical composition according to the present invention can further also include one or more other pharmaceutically active ingredient selected from the group consisting of an antibacterial agent, an antiviral agent, an anti-fungal agent, an anti-parasitic agent, an anti-inflammatory agent, an analgesic agent and an anti-pruritic agent.

Thus, there is further provided:

(I) a pharmaceutical composition including a selective DOR antagonist, as hereinbefore defined and another therapeutic agent, in admixture with a pharmaceutically-acceptable adjuvant, diluent or carrier; or
(II) a kit of parts comprising components:
(i) a pharmaceutical composition including a selective DOR antagonist (e.g. naltrindole), as hereinbefore defined, in admixture with a pharmaceutically-acceptable adjuvant, diluent or carrier; and
(ii) a pharmaceutical composition including at least one other therapeutic agent (e.g. a DOR agonist, a selective ligand for sensory receptor or another active agent), in admixture with a pharmaceutically-acceptable adjuvant, diluent or carrier,
which components (i) and (ii) are each provided in a form that is suitable for administration in conjunction with the other.

DOR antagonists of the present invention and DOR agonists and/or such other pharmaceutically active ingredients can be administered together as part of the same topical pharmaceutical composition or can be administered separately as part of an appropriate dose regimen designed to obtain the benefits of the combination therapy. The appropriate dose regimen, the amount of each dose administered, and specific intervals between doses of each pharmaceutically active agent will depend upon the specific combination of active agents employed, the condition of the subject being treated, and other factors.

The delivery of drugs through the topical application, for example on the skin, provides many advantages over other routes of administration. Primarily, topical drug delivery is a comfortable, convenient, and noninvasive way of administering drugs. The variable rates of absorption and metabolism associated with oral administration are avoided, as are other inherent inconveniences such as gastrointestinal irritation and the like. Topical drug delivery also makes possible a high degree of control over blood concentrations of any particular drug and allows for consistent drug delivery. These advantages enhance patient compliance and improve the safety and efficacy of medications.

The pharmaceutical compositions according to the present invention are also useful for promoting surface wound healing, minimizing scarring and encouraging cosmetically acceptable scar formation without dysaesthesia, improving the aesthetics of post-operative scars, facilitating and enhancing healing in burned and/or traumatized tissues, and reducing stretch marks.

The pharmaceutical compositions of the present invention are also useful for treating skin aging which includes methods of ameliorating symptoms of skin aging including wrinkles, sun damage, skin discoloration, rosacea, photoaging, lentigines, facial skin tightening including eyelids and jowls, dry and itchy skin and improvement of skin and blood vessel fragility, but also disorders of skin appendages (e.g. hair, nails, sebaceous and sweat glands) and of pigmentation (hyper-, hypo-, and depigmentations). More in particular the pharmaceutical compositions of the present invention also include methods of ameliorating symptoms of skin aging including wrinkles, sun damage, skin discoloration, rosacea, photoaging, lentigines, facial skin tightening including eyelids and jowls, dry and itchy skin and improvement of skin and blood vessel fragility.

The topical pharmaceutical compositions according to the present invention can be, but are not limited to, a solution, a cream, a patch, a gel, an ointment, a lotion, a tincture, a spray, a mousse, a cleansing composition, a powder, a tape, a vapor or a foam.

Devices for topical administration of drugs generally fall into either the category of liquid reservoir patches or of matrix patches. In liquid reservoir patches, the drug is stored as a liquid in a reservoir from which it diffuses to the skin. The patch includes a boundary layer that may include a rate-controlling membrane to control the release rate of the drug. In matrix patches, the drug is stored in a polymeric matrix that can be made of one or more layers for storing the drug, controlling the rate of release, and adhering to the skin. Liquid reservoir patches are easier to develop than matrix patches because of fewer problems such as incompatibility of drug and polymeric materials. Matrix patches, however, are easier to manufacture than liquid reservoir patches and are more comfortable and convenient to wear.

One embodiment of the device of the present invention is a patch for application to the skin of a subject. The patch has a skin-contacting matrix adhesive layer laminated or otherwise attached to a backing layer. Typically, the matrix adhesive layer is covered by a removable release liner before use to protect the matrix adhesive surface and keep it clean until it is applied to the skin. The backing layer acts as a support for the matrix adhesive layer and provides a barrier layer that prevents loss of the drug in the matrix adhesive layer to the environment. The material chosen for the backing should be compatible with the adhesive, drug, and permeation enhancer, and should be minimally permeable to any components of the patch. The backing can be opaque to protect components of the matrix patch from degradation from exposure to ultraviolet light. Further, the backing should be capable of binding to and supporting the adhesive layer, yet should be pliable to accommodate the movements of a person using the patch. Suitable materials for the backing include metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. A thickness of about 0.0127 to 0.254 millimeters can, for example, be used. As is known in the art, adhesive monomers can include carboxylic acid moieties (or salts thereof) and/or other functional groups, such as hydroxyl. Or, adhesive monomers may have no functional monomers (as synthesized, assuming no substantial hydrolysis of, for example, ester linkages). Adhesive polymers are often crosslinked to some degree, such as by use of crosslinking monomer. Useful adhesives include, for example, acrylics (e.g., polyacrylates including alkyl acrylates), polyvinyl acetates, natural and synthetic rubbers, ethylenevinylacetate copolymers, polysiloxanes, polyurethanes, plasticized polyether block amide copolymers, plasticized styrene-butadiene rubber block copolymers, and mixtures thereof. Polyacrylates can be, for example, Duro-Tak 87-4098, Duro-Tak 87-2052, Duro-Tak 387-2353 (or Duro-Tak 87-2353), Duro-Tak 387-2287 (or Duro-Tak 87-2287), Duro-Tak 387-2516 (or Duro-Tak 87-2516) (all from National Starch & Chemical, Bridewater, N.J.), or mixtures thereof. Styrene-butadiene rubber pressure sensitive adhesive can be, for example, DURO-TAK® 87-6173 adhesive (National Starch & Chemical). The release liner can be made of the same materials as the backing, or other suitable films coated with an appropriate release surface.

The patch can further comprise various additives in addition to the adhesive and permeation enhancer. These additives are generally those pharmaceutically acceptable ingredients that are known in the art of drug delivery and, more particularly, in the art of topical drug delivery. Nonlimiting examples of additive ingredients include diluents, excipients, emollients, plasticizers, skin irritation reducing agents (which can also include agents that reduce irritation to mucosa), carriers, and mixtures of these. For example, suitable diluents can include mineral oil, low molecular weight polymers, plasticizers, and the like. Some topical drug delivery formulations have a tendency to cause irritation after prolonged exposure to the skin, thus addition of an irritation reducing agent aids in achieving a composition that is better tolerated by the skin or mucosa.

The topical pharmaceutical compositions of the present invention can be also in the form of a suspension or dispersion in solvents or fatty substances, or alternatively in the form of an emulsion or micro emulsion, PET-emulsions, multiple emulsions, bickering emulsions, hydrogels, alcoholic gels, lipogels, one or multiphase solutions or a vesicular dispersion and other usual compositions, which can also be applied by pens, as masks or as sprays. The emulsions can also contain anionic, nonionic, cationic or amphoteric surfactant(s).

The topical pharmaceutical compositions of the invention can also contain usual pharmaceutical adjuvants and additives, such as preservatives/antioxidants, fatty substances/oils, water, organic solvents, silicones, thickeners, softeners, emulsifiers, sunscreens, antifoaming agents, moisturizers, fragrances, surfactants, fillers, sequestering agents, anionic, cationic, nonionic or amphoteric polymers or mixtures thereof, propellants, acidifying or basifying agents, dyes, colorants, pigments or nanopigments, e.g. those suited for providing a photoprotective effect by physically blocking out ultraviolet radiation, or any other ingredients usually formulated into topical pharmaceutical compositions. The necessary amounts of the dermatological adjuvants and additives can, based on the desired product, easily be chosen by a person skilled in the art. An additional amount of antioxidants/preservatives is generally preferred. Based on the invention all known antioxidants usually formulated into topical pharmaceutical compositions can be used.

Typically topical pharmaceutical compositions also contain surface active ingredients like emulsifiers, solubilizers and the like. An emulsifier enables two or more immiscible components to be combined homogeneously. Moreover, the emulsifier acts to stabilize the composition.

The lipid phase of the topical pharmaceutical compositions can advantageously be chosen from: mineral oils and mineral waxes; oils such as triglycerides of caprinic acid or caprylic acid and castor oil; oils or waxes and other natural or synthetic oils, in a preferred embodiment esters of fatty acids with alcohols e.g. isopropanol, propylene glycol, glycerin or esters of fatty alcohols with carboxylic acids or fatty acids; alkylbenzoates; and/or silicone oils such as dimethylpolysiloxane, diethylpolysiloxane, diphenylpolysiloxane, cyclomethicones and mixtures thereof.

Exemplary fatty substances which can be incorporated in the oil phase of the emulsion, microemulsion, oleo gel, hydrodispersion or lipodispersion of the topical pharmaceutical composition of the present invention are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Other fatty components suitable for use in the topical pharmaceutical compositions of the present invention include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils, apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicones (octamethylcyclotetrasiloxane; cetyldimethicone, hexamethylcyclotrisiloxane, polydimethyl-siloxane, poly(methylphenylsiloxane) and mixtures thereof.

The oily phase of the topical pharmaceutical compositions of the present invention can also contain natural vegetable or animal waxes such as bee wax, china wax, bumblebee wax and other waxes of insects as well as shea butter and cocoa butter.

A moisturizing agent may be incorporated into a topical pharmaceutical composition of the present invention to maintain hydration or rehydrate the skin. Moisturizers that prevent water from evaporating from the skin by providing a protective coating are called emollients. Additionally an emollient provides a softening or soothing effect on the skin surface and is generally considered safe for topical use. Preferred emollients include mineral oils, lanolin, petrolatum, capric/caprylic triglyceraldehydes, cholesterol, silicones such as dimeticone, cyclometicone, almond oil, jojoba oil, avocado oil, castor oil, sesame oil, sunflower oil, coconut oil and grape seed oil, cocoa butter, olive oil aloe extracts, fatty acids such as oleic and stearic, fatty alcohols such as cetyl and hexadecyl (ENJAY), diisopropyl adipate, hydroxybenzoate esters, benzoic acid esters of C₉₋₁₅-alcohols, isononyl iso-nonanoate, ethers such as polyoxypropylene butyl ethers and polyoxypropylene cetyl ethers, and C₁₂-i₅-alkyl benzoates, and mixtures thereof.

Moisturizers that bind water, thereby retaining it on the skin surface are called humectants. Suitable humectants can be incorporated into a topical pharmaceutical composition of the present invention such as glycerin, polypropylene glycol, polyethylene glycol, lactic acid, pyrrolidone carboxylic acid, urea, phospholipids, collagen, elastin, ceramides, lecithin sorbitol, PEG-4, and mixtures thereof.

The aqueous phase of topical pharmaceutical compositions of the present invention can contain the usual pharmaceutical additives such as alcohols, especially lower alcohols, preferably ethanol and/or isopropanol, low diols or polyols and their ethers, preferably propyleneglycol, glycerin, ethyleneglycol, ethyleneglycol monoethyl- or monobutylether, propyleneglycol monomethyl- or -monoethyl- or -monobutylether, diethyleneglycol monomethyl- or monoethylether and analogue products, polymers, foam stabilizers; electrolytes and especially one or more thickeners. However, preferably the topical pharmaceutical compositions of the present invention are free of ethanol, more preferably they are free of alcohols, and most preferably they are free of organic solvents, since such compounds can cause skin irritation.

Thickeners that may be used in topical pharmaceutical compositions of the present invention to assist in making the consistency of a product suitable include carbomer, siliciumdioxide, magnesium and/or aluminium silicates, beeswax, stearic acid, stearyl alcohol polysaccharides and their derivatives such as xanthan gum, hydroxypropyl cellulose, polyacrylamides, acrylate crosspolymers preferably a carbomer, such as Carbopole® of type 980, 981, 1382, 2984, 5984 alone or mixtures thereof.

Suitable neutralizing agents which may be included in the topical pharmaceutical composition of the present invention to neutralize components such as e.g. an emulsifier or a foam builder/stabilizer include but are not limited to alkali hydroxides such as a sodium and potassium hydroxide; organic bases such as diethanolamine (DEA), triethanolamine (TEA), aminomethyl propanol, and mixtures thereof; amino acids such as arginine and lysine and any combination of any foregoing.

The addition of electrolytes into the topical pharmaceutical composition of the present invention may be necessary to change the behavior of a hydrophobic emulsifier. Thus, the emulsions/microemulsions of this invention may contain preferably electrolytes of one or several salts including anions such as chloride, sulfates, carbonate, borate and aluminate, without being limited thereto. Other suitable electrolytes can be on the basis of organic anions such as, but not limited to, lactate, acetate, benzoate, propionate, tartrate and citrate. As cations preferably ammonium, alkylammonium, alkali- or alkaline earth metals, magnesium-, iron- or zinc-ions are selected. Especially preferred salts are potassium and sodium chloride, magnesium sulfate, zinc sulfate and mixtures thereof.

The cosmetic skincare composition of the invention may be for enhancing the appearance or odor of the body by improving at least one property of skin selected from the group consisting of wrinkles, elasticity, atrophy (thinning), texture, radiance, skin colour, tone and pigmentation and making skin fair, skin renewal, rejuvenation, reduction of pores and controls oily or dry skin, and pleasant skin feelings (e.g. warm or soft).

Alternatively, the cosmetic skincare composition of the invention may be for reducing at least one of wrinkling, elasticity, skin atrophy (thinning), improved skin texture and fair, radiant skin, skin renewal, reduction of pores, pigmentation of the skin.

Thus further aspects of the invention relate to the following.

(1) A selective DOR antagonist (e.g. naltrindole), as hereinbefore defined, and another therapeutic agent for use in the treatment of epithelial conditions, such as skin wounds, skin aging, skin tumors and/or skin sensation conditions.

In this aspect of the invention, selective DOR antagonist, as hereinbefore defined, may be administered sequentially, simultaneously or concomitantly with the other therapeutic agent.

(2) A selective DOR antagonist (e.g. naltrindole), as hereinbefore defined, for use in the treatment of epithelial conditions, such as skin wounds, skin aging, skin tumors and/or skin sensation conditions, wherein the selective DOR antagonist is administered sequentially, simultaneously or concomitantly with another therapeutic agent.
(3) Use of a selective DOR antagonist (e.g. naltrindole), as hereinbefore defined, and another therapeutic agent for the preparation of a medicament for the treatment of epithelial conditions, such as skin wounds, skin aging, skin tumors and/or skin sensation conditions, wherein the selective DOR antagonist is administered sequentially, simultaneously or concomitantly with the other therapeutic agent.
(4) Use of a selective DOR antagonist (e.g. naltrindole), as hereinbefore defined, for the preparation of a medicament for the treatment of epithelial conditions, such as skin wounds, skin aging, skin tumors and/or skin sensation conditions, wherein the medicament is administered in combination with another therapeutic agent.
(5) A method for treating epithelial conditions, such as skin wounds, skin aging, skin tumors and/or skin sensation conditions, which method comprises the administration of an effective amount of a selective DOR antagonist (e.g. naltrindole), as hereinbefore defined, and another therapeutic agent to a patient in need of such treatment.
(6) A combination product comprising
(A) a selective DOR antagonist (e.g. naltrindole), as hereinbefore defined, and
(B) another therapeutic agent,
wherein each of components (A) and (B) is formulated in admixture with a pharmaceutically-acceptable adjuvant, diluent or carrier.
   (7) A combination product as defined at (6) above for use in the treatment of epithelial conditions, such as skin wounds, skin aging, skin tumors and/or skin sensation conditions.
   (8) The use of a combination product as defined at (6) above for the manufacture of a medicament for the treatment of epithelial conditions, such as skin wounds, skin aging, skin tumors and/or skin sensation conditions.
   (9) A method of treatment of epithelial conditions, such as skin wounds, skin aging, skin tumors and/or skin sensation conditions, which method comprises the administration of an effective amount of a combination product as defined at (6) above.

When used herein, the term “another therapeutic agent” includes ligands for taste (TAS2R14) and ligands for olfactory receptors (OR2T4, OR11G2), and/or one or more therapeutic agents that are known to be useful for or be effective in the treatment of epithelial conditions, such as skin wounds, skin aging, skin tumors and/or skin sensation conditions.

The combination product described above provides for the administration of component (A) in conjunction with component (B), and may thus be presented either as separate formulations, wherein at least one of those formulations comprises component (A) and at least one comprises component (B), or may be presented (i.e. formulated) as a combined preparation (i.e. presented as a single formulation including component (A) and component (B)).

When used herein, the term “administered sequentially, simultaneously or concomitantly” may include:
administration of separate pharmaceutical formulations (one containing the selective DOR antagonist and one or more others containing the one or more the other therapeutic agents); and
administration of a single pharmaceutical formulation containing the selective DOR antagonist and the other therapeutic agent(s).

In the third aspect, the present invention provides a method for stimulating differentiation and proliferation of epithelial cells, comprising a step of contacting said cells with a selective DOR antagonist. Said differentiation and proliferation processes may be involved in skin homeostasis and wound healing.

Preferably, the selective DOR antagonist is selected from the group comprising naltrindole, methylnaltrindole, N,N(Me)₂-Dmt-Tic-OH, SoRI-9409, naltriben or pharmaceutically acceptable salts thereof.

Said method may further comprise a step of contacting said epithelial cells with a selective DOR agonist. Said selective DOR agonist is selected from the group consisting of SNC-80, BW373U86, DPI-287 and DPI-3290.

Said epithelial cells comprise all types of epithelial cells, including but not limited to skin cells and cells from mucosal, respiratory, gastro-intestinal epithelia. Said skin cells comprise such as keratinocytes, fibroblast, melanocyte, dendritic cells and skin immune cells.

The method according to the present invention may be used for stimulating nerve regeneration and/or endothelial homeostasis.

In a further aspect, the present invention provides a method for screening selective ligands for sensory receptor, comprising the steps of over-expressing a selective ligand for sensory receptor in epithelial cells, and screening the screening selective ligands for the sensory receptor.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

### In Vivo Assay—Wound Healing

The effects of selective DOR antagonist are tested in animal models of wound healing, preferably in mice, to determine whether a selective DOR antagonist improves the quality, rate or extent of wound healing.

In the present example, male mice of 3-4 months old were grouped into 5 groups and 10 in each group. Before experiment, mice are shaved one day before. Pentothal was used for anesthesia before experiments. Seven days experiments are used. Four wounds are made by cutting 6 mm diameter and 3 cm apart. Day 1 wound #1 for 7 day wound, Day 3 wound #2 for 5 day wound, Day 5 wound #3 for 3 day wound, Day 7 wound #4 for 1 day wound, Day 8 mice are sacrifice. A placebo cream or a cream containing one opioid ligand selected from DOR agonists (SNC 80, Dalargin [unspecific]), or unspecific DOR antagonist (naltrexone) and the selective (specific) DOR antagonist (Naltrindole) was directly applied to the wounds twice every day, morning and evening. At the end of experiments on Day 8, the biopsies will be taken from the mice and fixed in 4% formaldehyde and embedded in paraffin block. 10 animals were tested for each substances comparing different opioid ligands at the same concentration (1% w/w) including DOR agonist (SNC 80, Dalargin [unspecific]), or unspecific DOR antagonist (naltrexone) and the selective (specific) DOR antagonist (Naltrindole).

As can be seen from the results, only the specific DOR antagonist Naltridole improved the wound healing by 3 days. (FIGS. 1 and 2)

### Example 2

### In Vivo Assay—Skin Aging

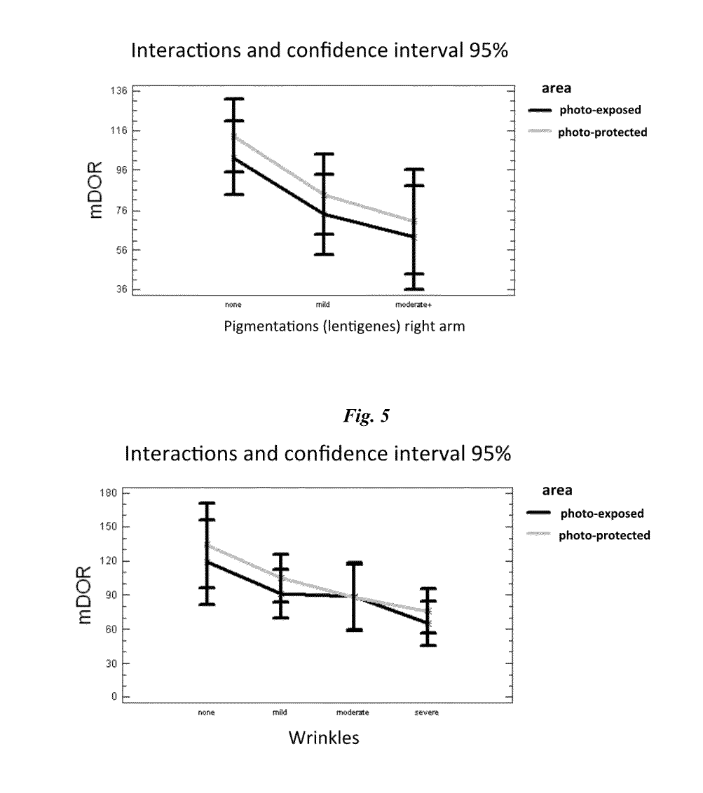

Data from Chinese women study show the down-regulation of epidermal DOR expression in women associated with age (FIG. 3), lentigines [taches] on the arms (FIG. 4) and wrinkle [rides] formation (FIG. 5). The epidermal expression of DOR is significantly reduced in aging process. DOR changes with age (older means less DOR) independent of sun-exposure (esp. >50 years). There is a negative correlation between DOR and typical signs of aging skin, development of lentigines (sun-spots) and wrinkles. There is also a negative correlation to melanin expression. The normal DOR expression is an indication for healthy, youthful skin (less wrinkles and less age spots).

Two biopsies were taken from the forearm extensor side (photo-exposed) and the inside of the upper arm (non-exposed). Before biopsy the areas are photo-documented for analysis of wrinkles and lentigines.

DOR expression is not different in photo-exposed and non-exposed skin (in all age classes) (FIG. 3). However, the epidermal DOR expression is significantly decreased in aged women compared to young woman. This reduction of DOR is clearly age-related and not to sun-damage of the skin.

| | 20-30_{(/5)} | 30-40_{(/6)} | 40-50_{(/6)} | 50-60_{(/5)} | 60-70_{(/6)} | p_tps |
|---|---|---|---|---|---|---|
| Exposed | 108.3 B | 90.9 AB | 99.0 B | 61.3 A | 58.6 A | S |
| | | | | | | (<0.01) |
| Protected | 115.8 B | 106.2 B | 113.6 B | 53.7 A | 75.2 AB | S |
| | | | | | | (=0.03) |
| p_zone | NS | NS | NS | NS | NS | |

Lentigines and DOR expression (FIG. 4): The amount of lentigines in the photos from the biopsied areas is correlated to epidermal DOR expression. More lentigines are present, lower is epidermal DOR expression.

| | | | | | Moderate | | | |
|---|---|---|---|---|---|---|---|---|
| | None (0) | | Mild (1-2) | | (3-4) | | p_lentigines | |
| Exposed | 102.2 | B | 73.9 | A | 62.4 | A | S | (=0.02) |
| Protected | 113.2 | B | 83.8 | AB | 70.2 | A | NS | (=0.06) |

Decrease of DOR expression is correlated with increase of lentigines (especially photo-exposed skin).

Wrinkles and DOR expression (FIG. 5): The amount of wrinkles in the photos from the biopsied areas is correlated to epidermal DOR expression.

| | | | | | Moderate | | | | | |
|---|---|---|---|---|---|---|---|---|---|---|
| | None (0) | | Mild (1-2) | | (3-4) | | Severe (≧5) | | | |
| | (n = 3) | | (n = 9) | | (n = 5) | | (n = 11) | | p_wrinkles | |
| Exposed | 118.9 | B | 90.7 | AB | 88.9 | AB | 65.6 | A | S | (=0.02) |
| Protected | 133.9 | | 104.7 | | 87.9 | | 75.8 | | NS | (=0.10) |

Decrease of DOR expression is correlated with increase of wrinkles, especially in photo-exposed skin.

### Example 3

### In Vitro Functional Assays

In-vitro regulation of DOR expression (mRNA, Protein) by selective DOR antagonist Naltrindole in cultured primary skin cells (keratinocytes, fibroblasts, melanocytes).

A: Semiquantification of DOR mRNA by Real Time PCR

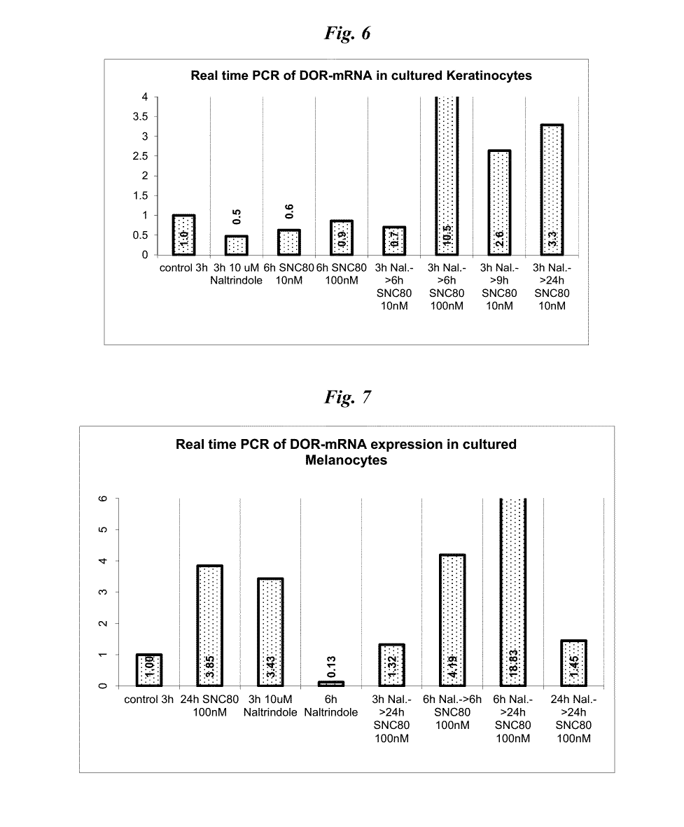

It is important to mention, that human cultured melanocytes and keratinocytes express more MOR than DOR mRNA. However, fibroblasts express more DOR than MOR mRNA. The results show (FIGS. 6, 7, 8), that mRNA of DOR is massively up-regulated if the human keratinocytes and melanocytes are pre-incubated for 3 hours with 10 μM Naltrindole and then exposed to the specific DOR agonist SNC-80. These data show that an interaction between antagonist and agonist is important for the regulation of DOR mRNA in human skin cells.

### B: Protein Expression of DOR by Western Blot Analysis:

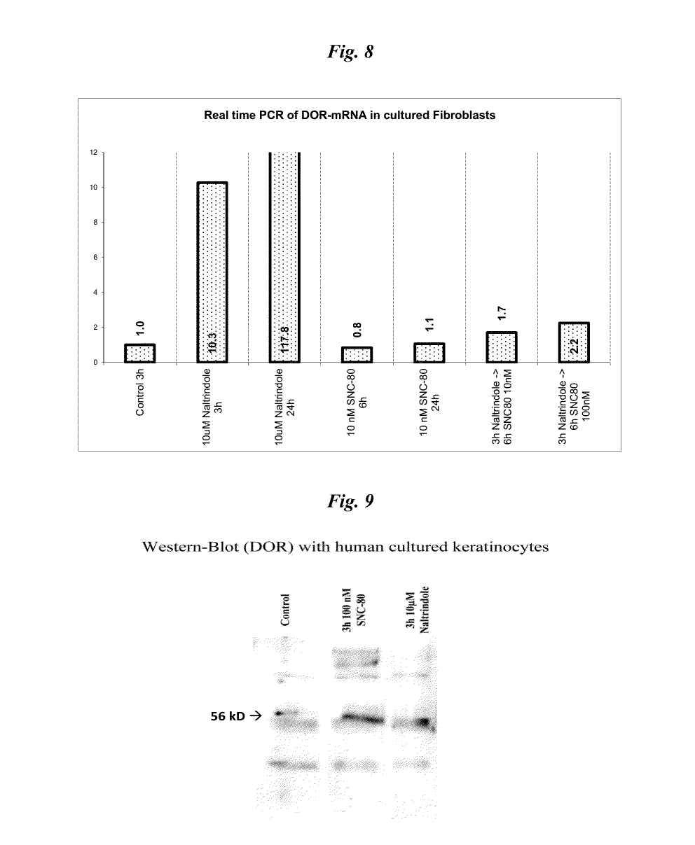

Human cultured keratinocytes (FIG. 9): The western blot shows an important up-regulation of DOR expression after exposure to specific DOR agonist (SNC-80) and antagonist (Naltrindole).

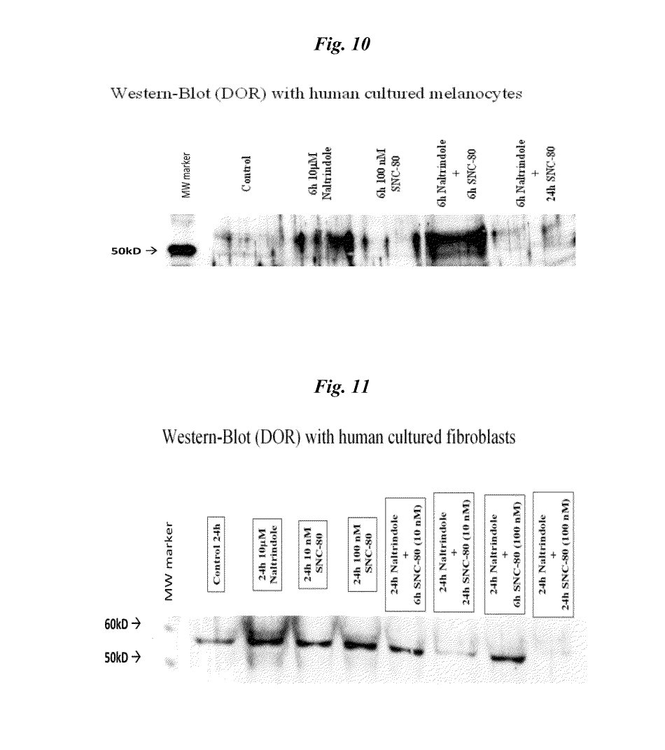

Human cultured melanocytes (FIG. 10): The western blot shows an important up-regulation of DOR expression after 6 h exposure to specific DOR antagonist (Naltrindole) but not to SNC-80. The combination of antagonist and agonist can upregulate the protein as well, however there seems to exist a delicate balance between agonist and antagonist.

Human cultured fibroblasts (FIG. 11): The western blot analysis shows an important upregulation of DOR expression after 24 h exposure to specific DOR antagonist (Naltrindole) and some up- and downregulation by specific DOR agonists (SNC 80) and some of their combinations.

### Example 4

### pERK Activation Protocol (Selectivity Functional Test)

Primary keratinocytes were grown to 70% and replaced with supplement free medium 16-24 hours before the experiments. The strongly stimulating supplement (25 mg/l bovine pituitary extract and 2.5 ug/l epidermal growth factor, EGF) in complete KFSM (Gibco) medium was used as positive control. Cells were exposed to SNC 80 for 15 min, DMSO (blank control) 15 min, or FCS/complete K-SFM for 30 min at 37° C. To block DOR signaling, cells were pre-incubated with Naltrindole for 5 min before exposure to SNC 80. Cells were lysed in 100 μl RIPA buffer and cell lysates frozen in liquid nitrogen.

Equal amount of protein were separated by electrophoresis over 10% polyacrylamid gels followed by protein transfer onto nitrocellulose membrane. Blots were transferred to primary antibody solution (anti-phospho ERK1/2 in 5% BSA/TBS-T) and incubated at 4° C. over night. After appropriate washing in TBS-T blots were incubated in secondary antibody (anti-rabbit Alexa-680 in 5% milk/TBS-T) for 45 min at room temperature in dark. Blots were washed 3× in TBS-T protected from light. Blots were imaged with the IR imager in 700 nm channel in a scan at 169 μm resolution. The results are shown in FIGS. 12a and 12b.

As can be seen from the figures, Naltrindole has effect close to 80% of these potent supplement effects and 3.4 times of control activity.

### Example 5

### Over-Expression of DOR in Human Keratinocytes Inhibits the Differentiation and Proliferation

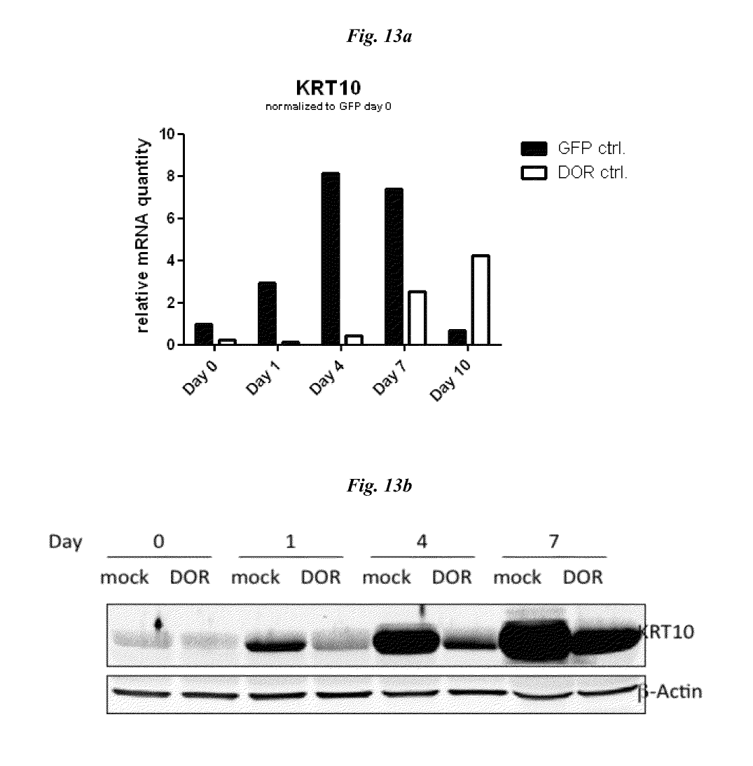

Over-expression of the DOR in primary human keratinocytes, immortalized keratinocytes N/TERTs reduces significantly the expression of Cytokeratin 10 (KRT10) on mRNA (FIG. 13a) and protein (FIG. 13b). Cytokeratin 10 is a typical marker for keratinocyte differentiation. This observation correlates with the significant over-expression of Cytokeratin 10 in DOR knockout mice (as published earlier).

POU2F3 is the key transcription factor regulating KRT10 and sensory function of keratinocyte: As can be seen from FIG. 14, until day 3 POU2F3 is down-regulated to promote cell proliferation function and later it is up-regulated to promote nerve regeneration and sensory restoration.

In addition, not only the differentiation of the N/TERT keratinocytes was affected by the DOR system, but also the proliferation. The proliferation of N/TERT keratinocytes after over-expression of the DOR receptor was monitored in Incucyte Device.

As can be seen from FIG. 15, the light blue line shows DOR over-expressing cells without exposure to ligand and the dark blue line shows DOR over-expressing cells with exposure to specific DOR ligand SNC-80. The brown line shows proliferation in GFP control cells and the purple line GFP control cells after exposure to SNC-80. The results confirm the significant reduction of proliferation in N/TERT keratinocytes after over-expression of the DOR receptor.

FIG. 16 shows that human cultured primary fibroblasts express a functional DOR. These Fibroblasts reveal significant faster migration and wound closure in an in-vitro scratch wound model after exposure to TGF-beta1 and/or DOR antagonist Naltrindole. The DOR agonist SNC80 does not seem to have any stimulation of migration compared to the control cells in DMSO diluent. The stimulation of wound closure in fibroblasts by TGF-beta1 is not surprising. However, the stimulation by the DOR-Antagonist Naltrindole was not expected. In an additional experiment we tested the effect of various specific opioid receptor antagonists on the wound closure/migration of human primary fibroblasts using the in-vitro wound scratch assay. These experiments suggest that the antagonism of the DOR₂ receptor is the most effective one, because the specific DOR₂ antagonist Naltriben is more effective than the mixed DOR_{1/2} antagonist Naltrindole and much more effective than the DOR₁ antagonist BNTX (Benzylidenenaltrexone). The specific MOR antagonist Cyprodime and the irreversible blocker of MOR (>KOR>>DOR) had no significant effects compared to the control without any antagonist.

These new data prove again that the DOR system is involved in skin differentiation, migration and homeostasis; in keratinocytes (Epidermis) as well as in fibroblasts (Dermis). All of these functions are crucial for a normal wound healing, skin homeostasis, but are also important for the homeostasis of skin appendages, especially hair.

### Example 6

### 3D Organotypic Skin Cultures Using Keratinocytes Overexpressing DOR

The DOR was over-expressed in immortalized human keratinocytes (N/TERT) using a specific Lentivirus expression system. As control we used a GFP expressing control (without the DOR gene) and the wild-type N/TERT keratinocytes.

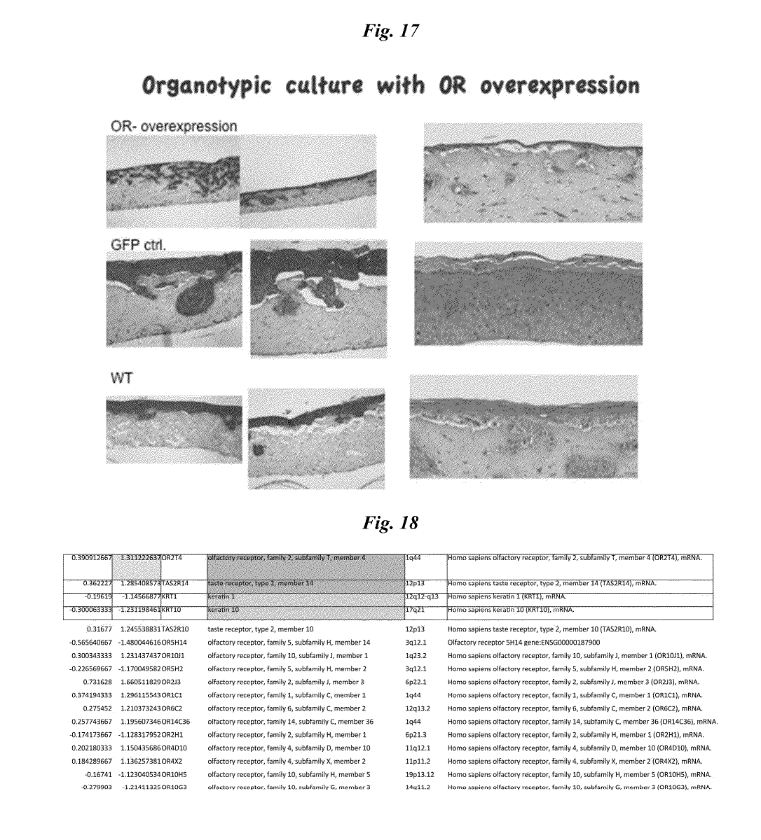

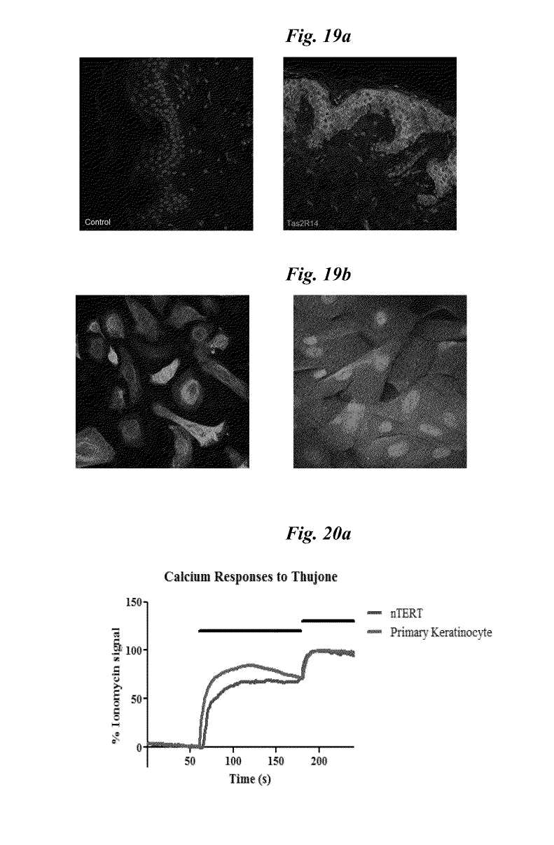

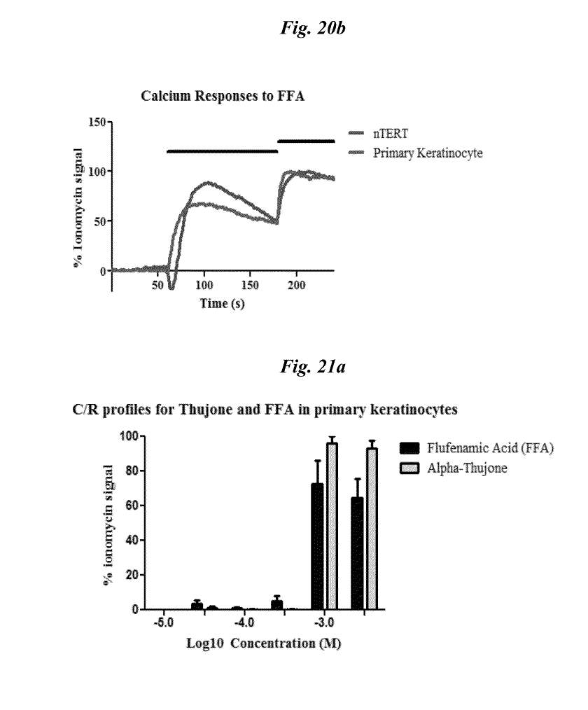

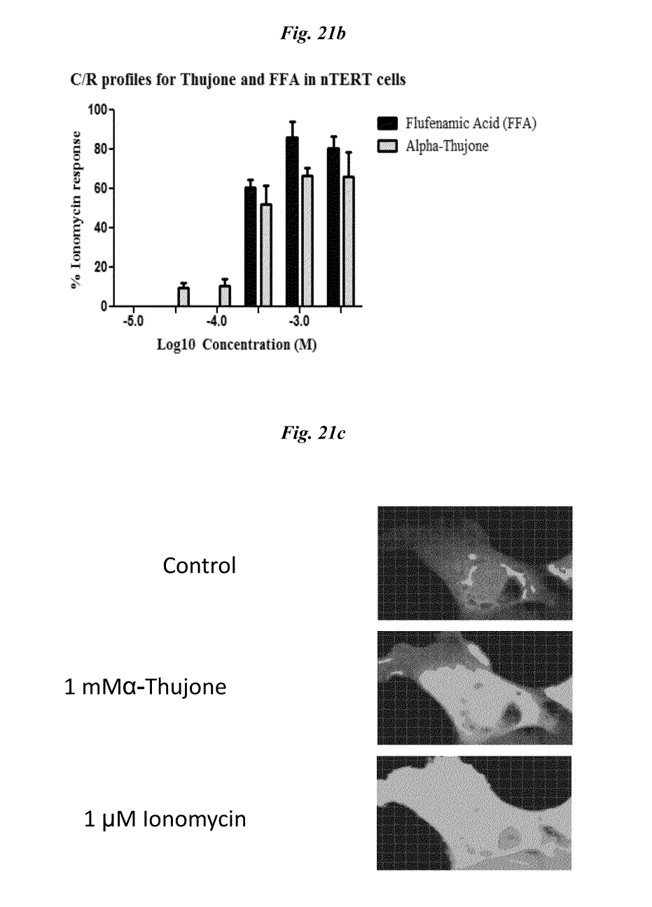

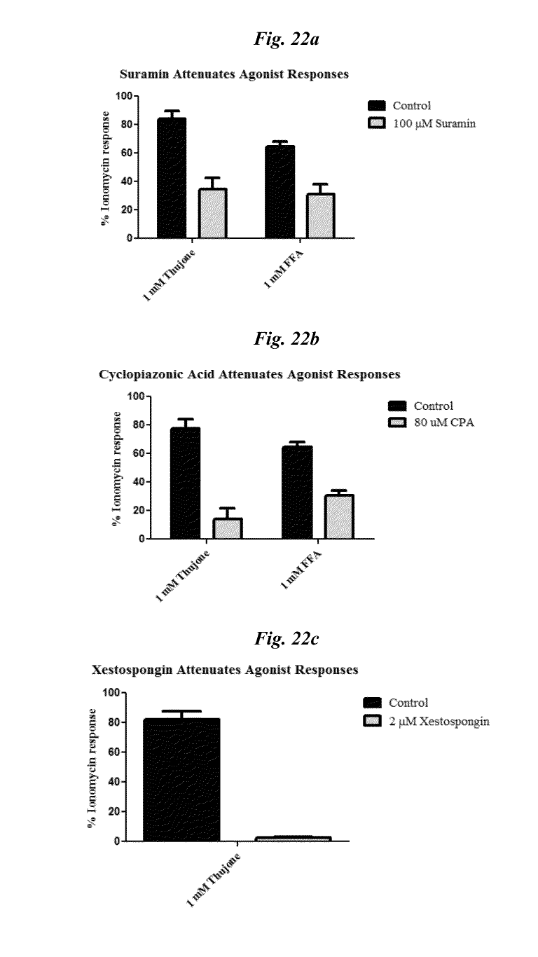

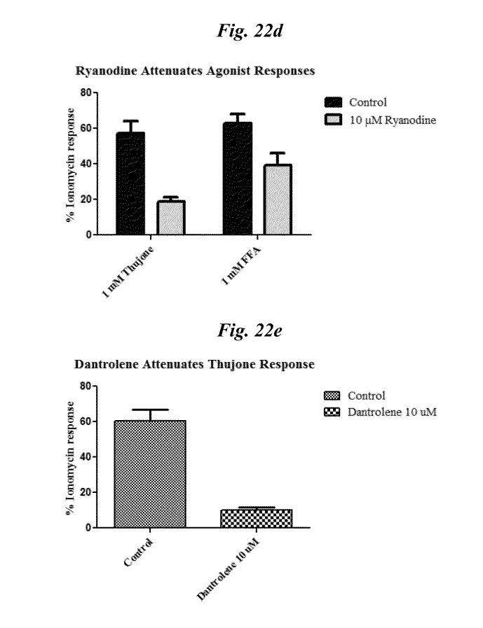

The pictures in FIG. 17 show clearly that the N/TERTs over-expressing DOR have an atrophic epidermis with no signs of differentiation (corneal layer) and a scirrhous infiltration pattern into the dermis. The GFP control and wild-type N/TERTs show a well organized epidermis of 6-10 cell layers and some focal formation of corneal layer. The organotypic culture experiments prove that the skin with over-expression of DOR has a totally changed phenotype. These 3D culture experiments prove again that the DOR system is indeed involved in skin homeostasis and finally also in wound healing.

### Example 7

### Functional Tas2R14 Expression in Skin Cells and its Impact on Sensory Transduction in the Skin

Sensory perceptions encompass a broad range of sensory modalities such as heat, cold, pain, itch and other physiological or pathological responses to stimuli. Based on the experiments by overexpressing DOR in human keratinocyte cell lines (HaCat cells) and performing a microarray analysis we found that the keratinocytes express specific taste (TAS2R14, TAS2R10) and various olfactory receptors (see FIG. 19). Further verification by real time PCR in various different keratinocytes (primary and N/TERT) proved the expression of these genes in skin.

Therefore, we advanced our studies and investigated the role of skin in sensation of bitter tasting compounds. This is potentially very interesting especially by recognizing that the skin can indeed sense bitter tasting chemicals that contact the skin. Many of these bitter tasting chemicals have been correlated with danger signals in various epidermal tissues and therefore the skin is capable to bring these sensations (bitter, olfactory) through the Central Nervous system to cognition and finally to a pleasant or unpleasant sensation that has to be accepted or removed.

TAS2R14 is one of the better described bitter taste receptor variants and has been described in locations within the gastrointestinal system outside the mouth, including the liver and duodenum. A study by Behrens et al. (2004) found a lack of TAS2R14 mRNA expression in various human tissues including salivary gland, kidney, cerebellum and testis (strong expression was found in taste bud-containing tissue from the tongue). We show for the first time that TAS2R14 receptors are not only expressed in the skin and cultured primary skin cells, they also display highly specific functional activity in response to known agonists of TAS2R14 in skin cell cultures. This has allowed functional studies of TAS2R14 to be carried out in a native setting. In addition, the elusive functional connectivity between skin cells as transducers and neuronal cells as conduits for sensing has been established, shedding some light on the controversial mechanisms of sensory transduction through the skin.

### Expression of TAS2R14 in Skin and Skin Cells

It is likely that the epidermal cells of the skin may themselves act as sensors for chemical and physical stimuli. We therefore used molecular, immunohistochemical and immunocytochemical methods to examine the possible presence of TAS2R14 receptors in the human normal skin and in cultured human keratinocytes. There was a clear presence of TAS2R14 in most layers of the skin (excluding the corneal layer) indicated by the green staining (FIG. 19a, right panel). The absence of any green staining in the control skin section shows a good lack resistance to non-specific staining by the antibodies (FIG. 19a, left panel). Cultured human primary keratinocytes also showed strong staining with the TAS2R14 antibody both in the undifferentiated state (FIG. 19b, left panel) and 7 days post-differentiation with 1.2 mM calcium (FIG. 19b, right panel). It should be noted that after differentiation, the antibody staining suggested a more diffuse and cytoplasmic location for the receptors (compared to a more membrane bound staining in undifferentiated keratinocytes. However, the PCR results with various specific primers in FIG. 23 suggests that the TAS2R14 and TAS2R10 have a different and specific expression pattern and especially TAS2R14 seems to be more expressed in highly proliferative, undifferentiated cells such as stem cells (H7). This indicate that TAS receptors are sensing and modulating cell growth, proliferation and differentiation.

### Functional TAS2R14 Responses in Skin Cells

TAS2R14 is known to be a G-protein coupled receptor, which can generate robust increases in intracellular free calcium in response to agonist applications and has been extensively characterized for agonists in expression systems (Behrens et al., 2004). Using the Fluo4-AM calcium indicator, we measured the changes in intracellular free calcium in response to bath application of the known TAS2R14 activators α-thujone (thujone) and flufenamic acid (FFA) in both primary keratinocytes and N/TERT cells (an immortalised human keratinocytes cell line; Dickson et al., 2000). Bath-application of 1 mM thujone (20a) or FFA (20b) resulted in robust increases in intracellular free calcium (measured by increase in fluorescence of fluo4) in both primary keratinocytes and in N/TERT cells (FIG. 20a). Maximal increases in fluorescence were generated in each recording by addition of the ionophore ionomycin at 1 μM, and calcium responses were calculated as percentages compared to maximal ionomycin signal. A concentration/response relationship existed to both compounds in both cell types, showing an almost all-or-nothing profile. In primary human keratinocytes thujone and FFA (21a) activated calcium signals significantly. Similar reaction has been seen in the human skin cell line, the N/TERT cells (21b). Under resting conditions the calcium appears condensed into the perinuclear region, and immediately following agonist application, the calcium response appears to spread from this perinuclear region into the surrounding cytoplasm, presumably following release from the ER stores. Ionomycin led to an immediate abundance of calcium throughout the cytoplasm (FIG. 21c).

### Intracellular Stores and G-Proteins are Involved in the Activation Pathway

Calcium activity induced by bitter taste receptor activation in the lingual epithelia appears related to activation of a specific G-protein (G α-Gustducin), although may involve other G-protein α-subunits (Caicedo et al., 2003). Suramin has been demonstrated to uncouple G-proteins indiscriminately, therefore attenuating receptor-G protein coupling (Chung & Kermode 2004), and so we used suramin to confirm the G protein coupling of the putative Tas2R14 responses observed in the skin cells. Calcium responses (expressed as percentage of ionomycin signal) to 1 mM thujone were significantly attenuated in the presence of 100 μM suramin (control: 84.2±5.5, 100 μM suramin: 34.4±8.2; p=0.002 unpaired t-test, n=4 all cases), as were FFA responses in N/TERT cells (control: 64.5±3.5, 100 μM suramin: 31.0±7.0; p=0.005 unpaired t-test, n=4 all cases)(FIG. 22a).

In addition, changes in intracellular free calcium in response to bitter compounds have been linked to signalling pathways releasing calcium from intracellular stores, in particular inositol triphosphate (IP3) (e.g. Yan et al., 2001).

Following this we assessed whether the calcium ATPase inhibitor cyclopiazonic acid (CPA), which has been previously shown to deplete endoplasmic reticulum (ER) calcium stores in cultured mouse keratinocytes (Li et al., 1995), affected the calcium responsiveness to Thujone or FFA. Calcium responses to both compounds were significantly attenuated in comparison to ionomycin (FIG. 22b). This result suggests that the intracellular calcium is indeed recruited from ER calcium stores.

A common route for G-protein signalling to release calcium from the ER is via the IP₃ pathway and so we used the IP3 receptor inhibitor Xestospongin C (Gafni et al., 1997) to block this potential mechanism. Xestospongin significantly attenuated the calcium response to application of thujone (% of ionomycin response (FIG. 22c), strongly suggesting that the IP3 pathway is involved in this specific signalling pathway in keratinocytes.

Another possible pathway of taste receptor signalling and calcium-induced calcium release from the ER is dependent on Ryanodine receptors. We tested this pathway by using Ryanodine and Dantrolene to assess calcium responses to Thujone after block of ryanodine receptors. Both Ryanodine (FIG. 22d) and Dantrolene (FIG. 22e) significantly attenuated Thujone induced calcium responses (FIG. 22d) Therefore, it seems that both the IP3 and Ryanodine pathway are specifically activated through the taste receptors in keratinocytes.

1. A method for treating skin wounds, skin aging, skin tumors and/or skin sensation conditions and/or for improving skin repair, comprising a step of administering an effective amount of:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor,
to a subject in need of the treatment.

2. The method of claim 1, wherein the method comprises a step of administering an effective amount of:
(a) a selective delta-opioid receptor (DOR) antagonist; or
(b) a combination of a selective DOR antagonist and an opioid receptor agonist; or
(c) a combination of a selective DOR antagonist and a selective ligand for a sensory receptor; or
(d) a combination of a selective DOR antagonist, an opioid receptor agonist and a selective ligand for a sensory receptor,
to a subject in need of the treatment.

3. The method of claim 2, wherein the method comprises a step of administering an effective amount of:
(a) a selective delta-opioid receptor (DOR) antagonist; or
(b) a combination of a selective DOR antagonist and an opioid receptor agonist,
to a subject in need of the treatment.

4. The method of any one of claims 1 to 3, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

5. The method of any one of claims 1 to 4, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

6. The method of any one of the preceding claims, wherein said selective DOR antagonist is naltrindole or a pharmaceutically acceptable salt and/or solvate thereof.

7. The method of any one of claims 1 to 5, wherein said selective DOR antagonist is a selective DOR₂ antagonist or a pharmaceutically acceptable salt and/or solvate thereof.

8. The method of claim 7, wherein said selective DOR₂ antagonist is naltriben, TIPPψ (H-Tyr-TicPsi[CH(2)NH]Phe-Phe-OH) or a pharmaceutically acceptable salt and/or solvate thereof.

9. The method of any one of claims 1 to 5, wherein said selective DOR antagonist is a selective DOR₁ antagonist or a pharmaceutically acceptable salt and/or solvate thereof, optionally wherein said selective DOR₁ antagonist is benzylidenenaltrexone or a pharmaceutically acceptable salt and/or solvate thereof.

10. The method of any one of claims 1 to 9, wherein said selective DOR₁ antagonist is benzylidenenaltrexone or a pharmaceutically acceptable salt and/or solvate thereof.

11. The method of any one of the preceding claims, wherein the method is for treating skin wounds, skin aging, skin tumors and/or skin sensation conditions.

12. The method of claim 11, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

13. The method of claim 11 or claim 12, wherein said opioid receptor agonist is a selective DOR agonist.

14. The method of claim 13, wherein the selective DOR agonist is selected from the group consisting of SNC-80, BW373U86, DPI-287 and DPI-3290 or pharmaceutically acceptable salts and/or solvates thereof.

15. The method of any one of claims 1 to 3, wherein the combination of a selective DOR antagonist and an opioid receptor agonist, or the combination of a selective DOR antagonist, an opioid receptor agonist and a selective ligand for a sensory receptor, comprises a mu-delta agonist-antagonist (MDAN) compound or pharmaceutically acceptable salts and/or solvates thereof.

16. The method of claim 15, wherein said MDAN compound comprises a mu opioid receptor (MOR) agonist linked to a DOR antagonist by a linker comprising a backbone of at least 16 atoms, or pharmaceutically acceptable salts and/or solvates thereof.

17. The method of claim 16, wherein said the MOR agonist linked to a DOR antagonist by a linker is oxymorphone or a derivative thereof.

18. The method of claim 17, wherein said MDAN compound has a general formula (I): 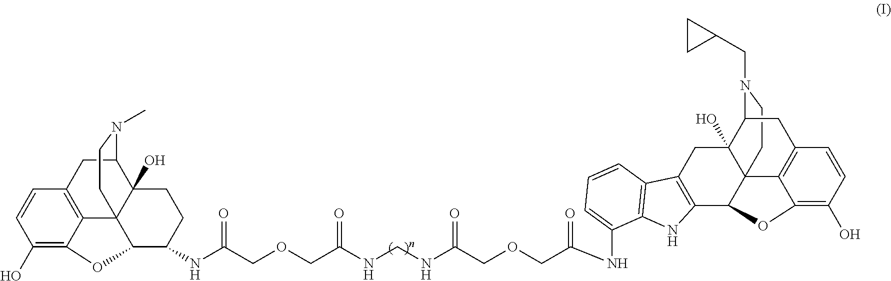
wherein n represents an integer of from 2 to 7, or pharmaceutically acceptable salts and/or solvates thereof.

19. The method of any one of the preceding claims, wherein said skin wounds, skin aging, skin tumors and/or skin sensation conditions are present in one or more of the mucosal epithelia, corneal epithelia, hair follicular epithelia, respiratory epithelia, gastro-intestinal epithelia, skin epithelia and skin appendages.

20. The method of claim 19, wherein the skin appendage is selected from one or more of the group consisting of hair follicles, sebaceous glands, sweat glands and nails.

21. The method of any one of the preceding claims, wherein said skin wound is caused by burns, chemical and/or mechanical injury to the skin.

22. The method of any one of the preceding claims, wherein said skin sensation conditions comprise at least one of pain, itch, tactile, burning, tickling, tingling, prickling, stinging, stretching, swelling, foreign body and sensitive skin.

23. The method of any one of the preceding claims, wherein said skin sensation conditions comprise at least one of itch, tactile, tickling, tingling, prickling, stretching, swelling, foreign body and sensitive skin.

24. The method of any one of the preceding claims, wherein the skin aging conditions are selected from the group consisting of wrinkles, skin discoloration/pigmentation, rosacea, senile angiomas, vessel fragility with haematomas, photo-aging, lentigines, loss of elasticity, increased fragility of skin, dry and itchy skin and delayed wound healing/repair.

25. The method of claim 24, wherein the skin pigmentation conditions comprise at least one disorder associated with hyper-, hypo- or depigmentation.

26. The method of any one of the preceding claims, wherein the method comprises a administering an effective amount of a selective delta-opioid receptor (DOR) antagonist or a combination of a selective DOR antagonist and a DOR agonist in an amount sufficient to induce pleasant sensations in the subject.

27. The method according to claim 26, wherein the pleasant sensations in the subject comprise at least one of sunlight, warmth, soft, euphoria, well-being, elation, happiness, excitement, and joy.

28. The method according to any one of the preceding claims, wherein the step of administering an effective amount of the combination of a selective DOR antagonist and an opioid receptor agonist, the combination of a selective DOR antagonist and a selective ligand for a sensory receptor, or the combination of a selective DOR antagonist, comprises simultaneous or concomitant administration of the selective DOR antagonist, the opioid receptor agonist and/or the selective ligand for a sensory receptor.

29. The method according to any one of claims 1 to 27, wherein the step of administering an effective amount of a combination of a selective DOR antagonist and an opioid receptor agonist, comprises sequential administration of the selective DOR antagonist and opioid receptor agonist.

30. The method according to claim 29, wherein the selective DOR antagonist is administered before the opioid receptor agonist.

31. The method according to claim 30, wherein the selective DOR antagonist is administered at least one minute before the opioid receptor agonist.

32. The method according to claim 31, wherein the selective DOR antagonist is administered between about 5 minutes to about 15 minutes before the opioid receptor agonist.

33. The method according to claim 31, wherein the selective DOR antagonist is administered from about one day to two days before the opioid receptor agonist.

34. The method according to any one of claims 28 to 33, wherein the administration of the selective DOR antagonist is capable of reducing or eliminating tolerance in the subject to the opioid receptor agonist.

35. The method of any one of the preceding claims, wherein the sensory receptor comprises a taste receptor and/or an olfactory receptor on a skin cell.

36. The method of claim 35, wherein the skin sensation conditions comprise sensitive skin.

37. The method of claim 35 or claim 36, wherein the selective ligand is thujone or flufenamic acid.

38. The method of any one of the preceding claims, further comprising administering one or more other pharmaceutical active ingredients selected from the group consisting of an anti-bacterial agent, an anti-viral agent, an anti-fungal agent, an anti-parasitic agent, an anti-inflammatory agent, an analgesic agent and an anti-pruritic agent, to the subject.

39. Use of a compound or combination in the preparation of a medicament for the treatment of skin wounds, skin aging, skin tumors and/or skin sensation conditions and/or for treatment to improve skin repair, wherein the treatment comprises a step of administering an effective amount of the compound or combination to a subject in need of such treatment, wherein the compound or combination is:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor.

40. The use of claim 39, wherein the compound or combination is:
(a) a selective delta-opioid receptor (DOR) antagonist; or
(b) a combination of a selective DOR antagonist and an opioid receptor agonist; or
(c) a combination of a selective DOR antagonist and a selective ligand for a sensory receptor; or
(d) a combination of a selective DOR antagonist, an opioid receptor agonist and a selective ligand for a sensory receptor.

41. The use of claim 40, wherein the compound or combination is:
(a) a selective delta-opioid receptor (DOR) antagonist; or
(b) a combination of a selective DOR antagonist and an opioid receptor agonist.

42. The use of any one of claims 39 to 41, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

43. The use of any one of claims 39 to 42, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

44. The use of any one of claims 39 to 43, wherein said selective DOR antagonist is naltrindole or a pharmaceutically acceptable salt and/or solvate thereof.

45. The use of any one of claims 39 to 43, wherein said selective DOR antagonist is a selective DOR₂ antagonist or a pharmaceutically acceptable salt and/or solvate thereof.

46. The use of claim 45, wherein said selective DOR₂ antagonist is naltriben, TIPPψ (H-Tyr-TicPsi[CH(2)NH]Phe-Phe-OH) or a pharmaceutically acceptable salt and/or solvate thereof.

47. The use of any one of claims 39 to 43, wherein said selective DOR antagonist is a selective DOR₁ antagonist or a pharmaceutically acceptable salt and/or solvate thereof, optionally wherein said selective DOR₁ antagonist is benzylidenenaltrexone or a pharmaceutically acceptable salt and/or solvate thereof.

48. The use of any one of claims 39 to 47, wherein the medicament is for the treatment of skin wounds, skin aging, skin tumors and/or skin sensation conditions.

49. The use of any one of claims 39 to 48, wherein said opioid receptor agonist is a mu opioid receptor (MOR) agonist, a DOR agonist or a pharmaceutically acceptable salt and/or solvate thereof.

50. The use of claim 49, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

51. The use of claim 49 or claim 50, wherein said opioid receptor agonist is a selective DOR agonist.

52. The use of claim 51, wherein the selective DOR agonist is selected from the group consisting of SNC-80, BW373U86, DPI-287 and DPI-3290 or pharmaceutically acceptable salts and/or solvates thereof.

53. The use of any one of claims 39 to 41, wherein the combination of a selective DOR antagonist and an opioid receptor agonist, or the combination of a selective DOR antagonist, an opioid receptor agonist and a selective ligand for a sensory receptor, comprises a mu-delta agonist-antagonist (MDAN) compound or pharmaceutically acceptable salts and/or solvates thereof.

54. The use of claim 53, wherein said MDAN compound comprises a mu opioid receptor (MOR) agonist linked to a DOR antagonist by a linker comprising a backbone of at least 16 atoms, or pharmaceutically acceptable salts and/or solvates thereof.

55. The use of claim 54, wherein said the MOR agonist linked to a DOR antagonist by a linker is oxymorphone or a derivative thereof.

56. The use of claim 55, wherein said MDAN compound has a general formula (I): 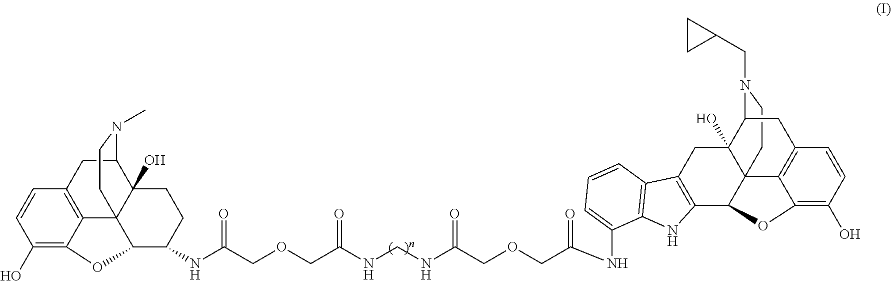
wherein n represents an integer of from 2 to 7, or pharmaceutically acceptable salts and/or solvates thereof.

57. The use of any one of claims 39 to 56, wherein said skin wounds, skin aging, skin tumors and/or skin sensation conditions are present in one or more of the mucosal epithelia, corneal epithelia, hair follicular epithelia, respiratory epithelia, gastro-intestinal epithelia, skin epithelia and skin appendages.

58. The use of claim 57, wherein the skin appendage is selected from one or more of the group consisting of hair follicles, sebaceous glands, sweat glands and nails.

59. The use of any one of claims 39 to 58, wherein said skin wound is caused by burns, chemical and/or mechanical injury to the skin.

60. The use of any one of claims 39 to 59, wherein said skin sensation conditions comprise at least one of pain, itch, tactile, burning, tickling, tingling, prickling, stinging, stretching, swelling, foreign body and sensitive skin.

61. The use of any one of claims 39 to 60, wherein said skin sensation conditions comprise at least one of itch, tactile, tickling, tingling, prickling, stretching, swelling, foreign body and sensitive skin.

62. The use of any one of claims 39 to 61, wherein the skin aging conditions are selected from the group consisting of wrinkles, skin discoloration/pigmentation, rosacea, senile angiomas, vessel fragility with haematomas, photo-aging, lentigines, loss of elasticity, increased fragility of skin, dry and itchy skin and delayed wound healing/repair.

63. The use of any one of claims 39 to 62, wherein the skin pigmentation conditions comprise at least one disorder associated with hyper-, hypo- or depigmentation.

64. The use of any one of claims 39 to 63, wherein the treatment comprises administering an effective amount of the compound or combination to the subject in an amount sufficient to induce pleasant sensations in the subject.

65. The use according to claim 64, wherein the pleasant sensations in the subject comprise at least one of sunlight, warmth, soft, euphoria, well-being, elation, happiness, excitement, and joy.

66. The use according to any one claims 39 to 65, wherein the step of administering an effective amount of the combination of a selective DOR antagonist and an opioid receptor agonist, the combination of a selective DOR antagonist and a selective ligand for a sensory receptor, or the combination of a selective DOR antagonist, comprises simultaneous or concomitant administration of the selective DOR antagonist, the opioid receptor agonist and/or the selective ligand for a sensory receptor.

67. The use according to any one of claims 39 to 65, wherein the step of administering an effective amount of the combination comprises sequential administration of the selective DOR antagonist, the opioid receptor agonist and/or the selective ligand for a sensory receptor.

68. The use according to claim 67, wherein the selective DOR antagonist is administered before the opioid receptor agonist and/or the selective ligand for a sensory receptor.

69. The use according to claim 68, wherein the selective DOR antagonist is administered at least one minute before the opioid receptor agonist and/or the selective ligand for a sensory receptor.

70. The use according to claim 69, wherein the selective DOR antagonist is administered between about 5 minutes to about 15 minutes before the opioid receptor agonist and/or the selective ligand for a sensory receptor.

71. The use according to claim 70, wherein the selective DOR antagonist is administered from about one day to two days before the opioid receptor agonist and/or the selective ligand for a sensory receptor.

72. The use according to any one of claims 66 to 71, wherein the administration of the selective DOR antagonist is capable of reducing or eliminating tolerance in the subject to the opioid receptor agonist.

73. The use of one of claims 66 to 72, wherein the sensory receptor comprises a taste receptor and/or an olfactory receptor on a skin cell.

74. The use of claim 73, wherein the skin sensation conditions comprise sensitive skin.

75. The use of claim 73 or claim 74, wherein the selective ligand is thujone or flufenamic acid.

76. The use of any one of claims 39 to 75, wherein the treatment further comprises administering one or more other pharmaceutical active ingredients selected from the group consisting of an anti-bacterial agent, an anti-viral agent, an anti-fungal agent, an anti-parasitic agent, an anti-inflammatory agent, an analgesic agent and an anti-pruritic agent, to the subject.

77. A compound or combination for use in the treatment of skin wounds, skin aging, skin tumors and/or skin sensation conditions and/or for treatment to improve skin repair, wherein the compound or combination is:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor, and
wherein the treatment comprises a step of administering an effective amount of the compound or combination to a subject in need of such treatment.

78. The compound or combination for use of claim 77, wherein the compound or combination is:
(a) a selective delta-opioid receptor (DOR) antagonist; or
(b) a combination of a selective DOR antagonist and an opioid receptor agonist; or
(c) a combination of a selective DOR antagonist and a selective ligand for a sensory receptor; or
(d) a combination of a selective DOR antagonist, an opioid receptor agonist and a selective ligand for a sensory receptor, and
wherein the treatment comprises a step of administering an effective amount of the compound or combination to a subject in need of such treatment.

79. The compound or combination for use of claim 78, wherein the compound or combination is:
(a) a selective delta-opioid receptor (DOR) antagonist; or
(b) a combination of a selective DOR antagonist and an opioid receptor agonist, and
wherein the treatment comprises a step of administering an effective amount of the compound or combination to a subject in need of such treatment.

80. The compound or combination for use of any one of claims 77 to 79, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

81. The compound or combination for use of any one of claims 77 to 80, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

82. The compound or combination for use of any one of claims 77 to 81, wherein said selective DOR antagonist is naltrindole or a pharmaceutically acceptable salt and/or solvate thereof.

83. The compound or combination for use of any one of claims 77 to 82, wherein said selective DOR antagonist is a selective DOR₂ antagonist or a pharmaceutically acceptable salt and/or solvate thereof.

84. The compound or combination for use of claim 83, wherein said selective DOR₂ antagonist is naltriben, TIPPψ (H-Tyr-TicPsi[CH(2)NH]Phe-Phe-OH) or a pharmaceutically acceptable salt and/or solvate thereof.

85. The compound or combination for use of any one of claims 77 to 84, wherein said selective DOR antagonist is a selective DOR₁ antagonist or a pharmaceutically acceptable salt and/or solvate thereof, optionally wherein said selective DOR₁ antagonist is benzylidenenaltrexone or a pharmaceutically acceptable salt and/or solvate thereof.

86. The compound or combination for use of any one of claims 77 to 85, wherein the compound or combination is for use in the treatment of skin wounds, skin aging, skin tumors and/or skin sensation conditions.

87. The combination for use of any one of claims 77 to 86, wherein said opioid receptor agonist is a mu opioid receptor (MOR) agonist, a DOR agonist or a pharmaceutically acceptable salt and/or solvate thereof.

88. The combination for use of claim 87, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

89. The combination for use of claim 87 or claim 88, wherein said opioid receptor agonist is a selective DOR agonist.

90. The combination for use of claim 89, wherein the selective DOR agonist is selected from the group consisting of SNC-80, BW373U86, DPI-287 and DPI-3290 or pharmaceutically acceptable salts and/or solvates thereof.

91. The compound or combination for use of any one of claims 77 to 79, wherein the combination of a selective DOR antagonist and an opioid receptor agonist, or the combination of a selective DOR antagonist, an opioid receptor agonist and a selective ligand for a sensory receptor, comprises a mu-delta agonist-antagonist (MDAN) compound or pharmaceutically acceptable salts and/or solvates thereof.

92. The compound or combination for use of claim 91, wherein said MDAN compound comprises a mu opioid receptor (MOR) agonist linked to a DOR antagonist by a linker comprising a backbone of at least 16 atoms, or pharmaceutically acceptable salts and/or solvates thereof.

93. The compound or combination for use of claim 92, wherein said the MOR agonist linked to a DOR antagonist by a linker is oxymorphone or a derivative thereof.

94. The compound or combination for use of claim 93, wherein said MDAN compound has a general formula (I): 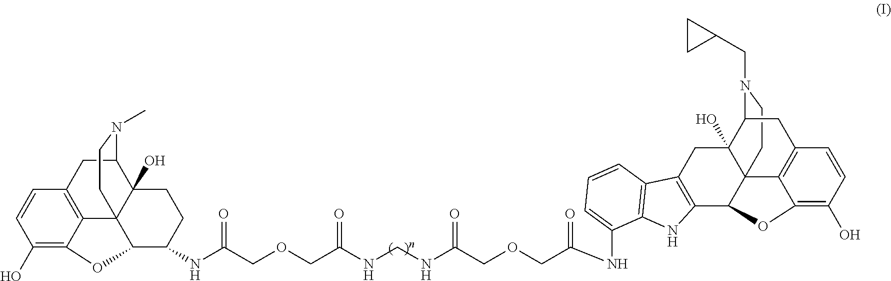
wherein n represents an integer of from 2 to 7, or pharmaceutically acceptable salts and/or solvates thereof.

95. The compound or combination for use of any one of claims 77 to 94, wherein said skin wounds, skin aging, skin tumors and/or skin sensation conditions are present in one or more of the mucosal epithelia, corneal epithelia, hair follicular epithelia, respiratory epithelia, gastro-intestinal epithelia, skin epithelia and skin appendages.

96. The compound or combination for use of claim 95, wherein the skin appendage is selected from one or more of the group consisting of hair follicles, sebaceous glands, sweat glands and nails.

97. The compound or combination for use of any one of claims 77 to 96, wherein said skin wound is caused by burns, chemical and/or mechanical injury to the skin.

98. The compound or combination for use of any one of claims 77 to 97, wherein said skin sensation conditions comprise at least one of pain, itch, tactile, burning, tickling, tingling, prickling, stinging, stretching, swelling, foreign body and sensitive skin.

99. The compound or combination for use of any one of claims 77 to 98, wherein said skin sensation conditions comprise at least one of itch, tactile, tickling, tingling, prickling, stretching, swelling, foreign body and sensitive skin.

100. The compound or combination for use of any one of claims 77 to 99, wherein the skin aging conditions are selected from the group consisting of wrinkles, skin discoloration/pigmentation, rosacea, senile angiomas, vessel fragility with haematomas, photo-aging, lentigines, loss of elasticity, increased fragility of skin, dry and itchy skin and delayed wound healing/repair.

101. The method of claim 100, wherein the skin pigmentation conditions comprise at least one disorder associated with hyper-, hypo- or depigmentation.

102. The compound or combination for use of any one of claims 77 to 101, wherein the treatment comprises a administering an effective amount of a selective delta-opioid receptor (DOR) antagonist or a combination of a selective DOR antagonist and a DOR agonist to the subject in an amount sufficient to induce pleasant sensations in the subject.

103. The compound or combination for use according to claim 102, wherein the pleasant sensations in the subject comprise at least one of sunlight, warmth, soft, euphoria, well-being, elation, happiness, excitement, and joy.

104. The combination for use according to any one claims 77 to 103, wherein the step of administering an effective amount of the combination of a selective DOR antagonist and an opioid receptor agonist, the combination of a selective DOR antagonist and a selective ligand for a sensory receptor, or the combination of a selective DOR antagonist, comprises simultaneous or concomitant administration of the selective DOR antagonist, the opioid receptor agonist and/or the selective ligand for a sensory receptor.

105. The combination for use according to any one of claims 77 to 103, wherein the step of administering an effective amount of the combination comprises sequential administration of the selective DOR antagonist, the opioid receptor agonist and/or the selective ligand for a sensory receptor.

106. The compound or combination for use according to claim 105, wherein the selective DOR antagonist is administered before the opioid receptor agonist and/or the selective ligand for a sensory receptor.

107. The compound or combination for use according to claim 106, wherein the selective DOR antagonist is administered at least one minute before the opioid receptor agonist and/or the selective ligand for a sensory receptor.

108. The compound or combination for use according to claim 107, wherein the selective DOR antagonist is administered between about 5 minutes to about 15 minutes before the opioid receptor agonist and/or the selective ligand for a sensory receptor.

109. The compound or combination for use according to claim 108, wherein the selective DOR antagonist is administered from about one day to two days before the opioid receptor agonist and/or the selective ligand for a sensory receptor.

110. The compound or combination for use according to any one of claims 77 to 109, wherein the administration of the selective DOR antagonist is capable of reducing or eliminating tolerance in the subject to the opioid receptor agonist.

111. The compound or combination for use of claim 110, wherein the sensory receptor comprises a taste receptor and/or an olfactory receptor on a skin cell.

112. The compound or combination for use of claim 111, wherein the skin sensation conditions comprise sensitive skin.

113. The compound or combination for use of claim 111 or claim 112, wherein the selective ligand is thujone or flufenamic acid.

114. The compound or combination for use of any one of claims 77 to 113, further comprising administering one or more other pharmaceutical active ingredients selected from the group consisting of an anti-bacterial agent, an anti-viral agent, an anti-fungal agent, an anti-parasitic agent, an anti-inflammatory agent, an analgesic agent and an anti-pruritic agent, to the subject.

115. A topical pharmaceutical composition for treating disorders of skin appendages and pigmentation, skin wounds, skin aging, skin tumors and/or skin sensation conditions and/or for treatment to improve skin repair, comprising an effective amount of:
(a) a selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor, and
a pharmaceutically acceptable adjuvant, diluent or carrier.

116. A topical pharmaceutical composition for treating disorders of skin appendages and pigmentation, skin wounds, skin aging, skin tumors and/or skin sensation conditions, comprising an effective amount of:
(a) a selective delta-opioid receptor (DOR) antagonist; or
(b) a combination of a selective DOR antagonist and an opioid receptor agonist; or
(c) a combination of a selective DOR antagonist and a selective ligand for a sensory receptor; or
(d) a combination of a selective DOR antagonist, an opioid receptor agonist and a selective ligand for a sensory receptor, and
a pharmaceutically acceptable adjuvant, diluent or carrier.

117. A topical pharmaceutical composition for treating disorders of skin appendages and pigmentation, skin wounds, skin aging, skin tumors and/or skin sensation conditions, comprising an effective amount of:
(a) a selective delta-opioid receptor (DOR) antagonist; or
(b) a combination of a selective DOR antagonist and an opioid receptor agonist,
and a pharmaceutically acceptable adjuvant, diluent or carrier.

118. The topical pharmaceutical composition of any one of claims 115 to 117, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

119. The topical pharmaceutical composition of any one of claims 115 to 118, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

120. The topical pharmaceutical composition of any one of claims 115 to 119, wherein said selective DOR antagonist is naltrindole or a pharmaceutically acceptable salt and/or solvate thereof.

121. The topical pharmaceutical composition of any one of claims 115 to 119, wherein said selective DOR antagonist is a selective DOR₂ antagonist or a pharmaceutically acceptable salt and/or solvate thereof.

122. The topical pharmaceutical composition of claim 121, wherein said selective DOR₂ antagonist is naltriben, TIPPψ (H-Tyr-TicPsi[CH(2)NH]Phe-Phe-OH) or a pharmaceutically acceptable salt and/or solvate thereof.

123. The topical pharmaceutical composition of any one of claims 115 to 119, wherein said selective DOR antagonist is a selective DOR₁ antagonist or a pharmaceutically acceptable salt and/or solvate thereof, optionally wherein said selective DOR₁ antagonist is benzylidenenaltrexone or a pharmaceutically acceptable salt and/or solvate thereof.

124. The topical pharmaceutical composition of any one of claims 115 to 123, wherein the topical pharmaceutical composition for treating disorders of skin appendages and pigmentation, skin wounds, skin aging, skin tumors and/or skin sensation conditions.

125. The topical pharmaceutical composition of any one of claims 115 to 124, wherein said opioid receptor agonist is a mu opioid receptor (MOR) agonist, a DOR agonist or a pharmaceutically acceptable salt and/or solvate thereof.

126. The topical pharmaceutical composition of claim 125, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

127. The topical pharmaceutical composition of claim 125 or claim 126, wherein said opioid receptor agonist is a selective DOR agonist.

128. The topical pharmaceutical composition of claim 127, wherein the selective DOR agonist is selected from the group consisting of SNC-80, BW373U86, DPI-287 and DPI-3290 or pharmaceutically acceptable salts and/or solvates thereof.

129. The topical pharmaceutical composition of any one of claims 115 to 117, wherein the combination of a selective DOR antagonist and an opioid receptor agonist, or the combination of a selective DOR antagonist, an opioid receptor agonist and a selective ligand for a sensory receptor, comprises a mu-delta agonist-antagonist (MDAN) compound or pharmaceutically acceptable salts and/or solvates thereof.

130. The topical pharmaceutical composition of claim 129, wherein said MDAN compound comprises a mu opioid receptor (MOR) agonist linked to a DOR antagonist by a linker comprising a backbone of at least 16 atoms, or pharmaceutically acceptable salts and/or solvates thereof.

131. The topical pharmaceutical composition of claim 130, wherein said the MOR agonist linked to a DOR antagonist by a linker is oxymorphone or a derivative thereof.

132. The topical pharmaceutical composition of claim 131, wherein said MDAN compound has a general formula (I): 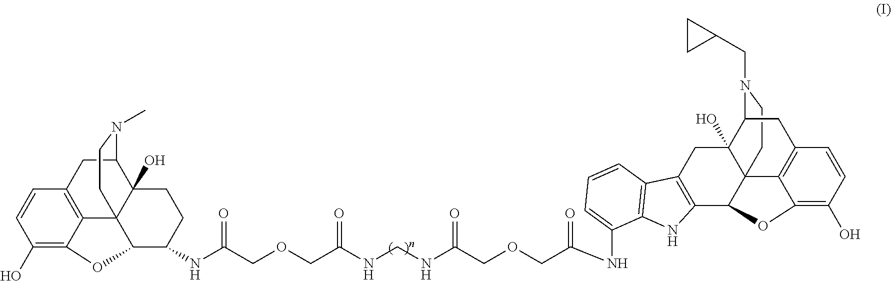
wherein n represents an integer of from 2 to 7, or pharmaceutically acceptable salts and/or solvates thereof.

133. The topical pharmaceutical composition of any one of claims 115 to 132, wherein said skin pigmentation conditions, skin wounds, skin aging, skin tumors and/or skin sensation conditions are present in one or more of the mucosal epithelia, corneal epithelia, hair follicular epithelia, respiratory epithelia, gastro-intestinal epithelia, skin epithelia and skin appendages.

134. The topical pharmaceutical composition of claim 133, wherein the skin appendage is selected from one or more of the group consisting of hair follicles, sebaceous glands, sweat glands and nails.

135. The topical pharmaceutical composition of any one of claims 115 to 134, wherein said skin wound is caused by burns, chemical and/or mechanical injury to the skin.

136. The topical pharmaceutical composition of any one of claims 115 to 135, wherein said skin sensation conditions comprise at least one of pain, itch, tactile, burning, tickling, tingling, prickling, stinging, stretching, swelling, foreign body and sensitive skin.

137. The topical pharmaceutical composition of any one of claims 115 to 136, wherein said skin sensation conditions comprise at least one of itch, tactile, tickling, tingling, prickling, stretching, swelling, foreign body and sensitive skin.

138. The topical pharmaceutical composition of any one of claims 115 to 137, wherein the skin aging conditions are selected from the group consisting of wrinkles, skin discoloration/pigmentation, rosacea, senile angiomas, vessel fragility with haematomas, photo-aging, lentigines, loss of elasticity, increased fragility of skin, dry and itchy skin and delayed wound healing/repair.

139. The method of claim 138, wherein the skin pigmentation conditions comprise at least one disorder associated with hyper-, hypo- or depigmentation.

140. The topical pharmaceutical composition of any one of claims 115 to 139, comprising the selective delta-opioid receptor (DOR) antagonist, DOR agonist and/or the selective ligand for a sensory receptor, in an amount sufficient to induce pleasant sensations in the subject.

141. The topical pharmaceutical composition according to claim 140, wherein the pleasant sensations in the subject comprise at least one of sunlight, warmth, soft, euphoria, well-being, elation, happiness, excitement, and joy.

142. The topical pharmaceutical composition of claim 142, wherein the sensory receptor comprises a taste receptor and/or an olfactory receptor on a skin cell.

143. The topical pharmaceutical composition of claim 143, wherein the skin sensation conditions comprise sensitive skin.

144. The topical pharmaceutical composition of claim 142 or claim 143, wherein the selective ligand is thujone or flufenamic acid.

145. The topical pharmaceutical composition of any one of claims 115 to 144, further comprising administering one or more other pharmaceutical active ingredients selected from the group consisting of an anti-bacterial agent, an anti-viral agent, an anti-fungal agent, an anti-parasitic agent, an anti-inflammatory agent, an analgesic agent and an anti-pruritic agent, to the subject.

146. A medical device for application of the topical pharmaceutical composition according to any one of claims 115 to 145, wherein the device is a dermal patch or a bandage comprising said topical pharmaceutical composition.

147. The medical device of claim 146, wherein the patch is a liquid reservoir patch or a matrix patch.

148. A kit comprising:
(A) a first topical pharmaceutical composition comprising a selective DOR antagonist and a pharmaceutically acceptable adjuvant, diluent or carrier; and
(B) a second topical pharmaceutical composition comprising at least one pharmaceutical active ingredient and a pharmaceutically acceptable adjuvant, diluent or carrier, and optionally
(C) instructions for the simultaneous, concomitant or sequential administration of the selective DOR antagonist of (A) and the at least one pharmaceutical active ingredient of (B), to a subject in need thereof,
wherein the at least one pharmaceutical active ingredient of (B) is selected from the group consisting of an anti-bacterial agent, an anti-viral agent, an anti-fungal agent, an anti-parasitic agent, an anti-inflammatory agent, an analgesic agent and an anti-pruritic agent, an opioid receptor agonist and a selective ligand for a sensory receptor.

149. The kit of claim 148, wherein the selective DOR antagonist of (A) and the at least one pharmaceutical active ingredient of (B) are for simultaneous or concomitant administration to the subject.

150. The kit of claim 148, wherein the selective DOR antagonist of (A) and the at least one pharmaceutical active ingredient of (B) are for sequential administration to the subject.

151. The kit of claim 150, wherein the selective DOR antagonist of (A) is for administration to the subject before the administration of the at least one pharmaceutical active ingredient of (B).

152. The kit of claim 151, wherein the selective DOR antagonist of (A) is for administration to the subject at least one minute before the administration of the at least one pharmaceutical active ingredient of (B).

153. The kit of claim 152, wherein the selective DOR antagonist of (A) is for administration to the subject between about 5 minutes to about 15 minutes before the administration of the at least one pharmaceutical active ingredient of (B).

154. The kit of claim 152, wherein the selective DOR antagonist of (A) is for administration to the subject from about one day to about two days before the administration of the at least one pharmaceutical active ingredient of (B).

155. The kit of claim 152, wherein the at least one pharmaceutical active ingredient of (B) comprises an opioid receptor agonist, and wherein the administration of the selective DOR antagonist of (A) to the subject is capable of reducing or eliminating tolerance in the subject to the opioid receptor agonist.

156. A method for modulating differentiation and/or proliferation of cells, comprising a step of contacting said cells with a selective DOR antagonist and/or a selective ligand for a sensory receptor.

157. The method of claim 156, wherein the method comprises stimulating differentiation of the cells.

158. The method of claim 156, wherein the cells are high-proliferative cells.

159. The method of claim 158, wherein the high-proliferative cell is an epithelial cell or a stem cell, optionally wherein the stem cell is not derived from a human embryonic stem cell.

160. The method of any one of claims 156 to 159, wherein the method is an in vitro method.

161. The method of any one of claims 156 to 160, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

162. The method of claim 161, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

163. The method of any one of claims any one of claims 156 to 162, wherein said selective DOR antagonist is naltrindole or a pharmaceutically acceptable salt and/or solvate thereof.

164. The method of any one of claims any one of claims 156 to 163, wherein said selective DOR antagonist is a selective DOR₂ antagonist or a pharmaceutically acceptable salt and/or solvate thereof.

165. The method of claim 164, wherein said selective DOR₂ antagonist is naltriben, TIPPψ (H-Tyr-TicPsi[CH(2)NH]Phe-Phe-OH) or a pharmaceutically acceptable salt and/or solvate thereof.

166. The method of any one of claims 156 to 165, wherein said selective DOR antagonist is a selective DOR₁ antagonist or a pharmaceutically acceptable salt and/or solvate thereof.

167. The method of claim 166, wherein said selective DOR₁ antagonist is benzylidenenaltrexone or a pharmaceutically acceptable salt and/or solvate thereof.

168. The method of any one of claims 156 to 167, wherein said method further comprises a step of contacting said epithelial cells with an opioid receptor agonist or a pharmaceutically acceptable salt and/or solvate thereof.

169. The method of claim 168, wherein said opioid receptor agonist is a MOR agonist or a selective DOR agonist or a pharmaceutically acceptable salt and/or solvate thereof.

170. The method of claim 169, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

171. The method of claim 168 or claim 169, wherein said opioid receptor agonist is a selective DOR agonist.

172. The method of claim 171, wherein the selective DOR agonist is selected from the group consisting of SNC-80, BW373U86, DPI-287 and DPI-3290 or pharmaceutically acceptable salts and/or solvates thereof.

173. The method of claim 168, wherein said method comprises a step of contacting said epithelial cells with a mu-delta agonist-antagonist (MDAN) compound or a pharmaceutically acceptable salts and/or solvate thereof.

174. The method of claim 173, wherein said MDAN compound comprises a mu opioid receptor (MOR) agonist linked to a DOR antagonist by a linker comprising a backbone of at least 16 atoms, or pharmaceutically acceptable salts and/or solvates thereof.

175. The method of claim 174, wherein said the MOR agonist linked to a DOR antagonist by a linker is oxymorphone or a derivative thereof.

176. The method of claim 175, wherein said MDAN compound has a general formula (I): 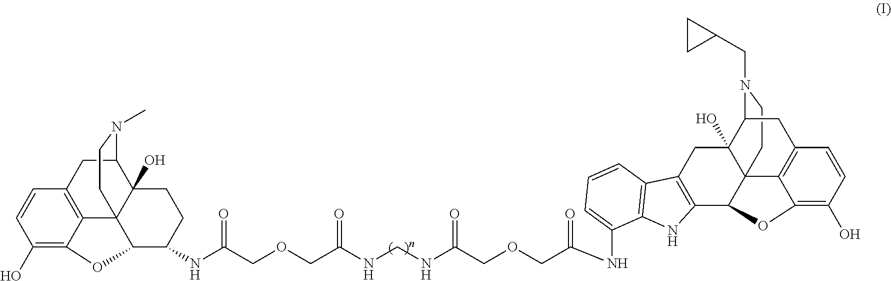
wherein n represents an integer of from 2 to 7, or pharmaceutically acceptable salts and/or solvates thereof.

177. The method of any one of claims 156 to 176, wherein said epithelial cells comprise skin cells and/or cells from mucosal, respiratory, gastro-intestinal epithelia, skin appendages.

178. The method of claim 177, wherein said epithelial cells comprises skin cells and/or cells from mucosal, respiratory, gastro-intestinal epithelia.

179. The method of claim 178, wherein said cells from skin appendages comprise cells from one or more of the group consisting of hair follicles, nail matrix, sweat glands and sebaceous glands.

180. The method of claim 178 or claim 179, wherein the skin cells comprise keratinocytes, fibroblast, melanocyte, dendritic cells and skin immune cells.

181. The method of any one of claims 156 to 180, wherein the method is for stimulating nerve regeneration and/or endothelial homeostasis.

182. The method of any one of claims 156 to 181, wherein the sensory receptor comprises a taste receptor and/or an olfactory receptor on a skin cell.

183. The method of any one of claims 156 to 182, wherein the selective ligand is thujone or flufenamic acid.

184. A cosmetic skin care composition comprising an effective amount of
(a) at least one selective delta-opioid receptor (DOR) antagonist; or
(b) a combination of a selective DOR antagonist and an opioid receptor agonist; or
(c) a selective ligand for a sensory receptor; or
(d) a combination of a selective DOR antagonist and a selective ligand for a sensory receptor; or
(e) a combination of a selective DOR antagonist, an opioid receptor agonist and a selective ligand for a sensory receptor, and
a cosmetically acceptable adjuvant, diluent or carrier.

185. The cosmetic skin care composition of claim 184, comprising an effective amount of
(a) at least one selective delta-opioid receptor (DOR) antagonist; or
(b) a combination of a selective DOR antagonist and an opioid receptor agonist; or
(c) a combination of a selective DOR antagonist and a selective ligand for a sensory receptor; or
(d) a combination of a selective DOR antagonist, an opioid receptor agonist and a selective ligand for a sensory receptor, and
a cosmetically acceptable adjuvant, diluent or carrier.

186. The cosmetic skin care composition of claim 185, comprising an effective amount of
(a) at least one selective delta-opioid receptor (DOR) antagonist; or
(b) a combination of a selective DOR antagonist and an opioid receptor agonist,
and a cosmetically acceptable adjuvant, diluent or carrier.

187. The cosmetic skin care composition of any one of claims 184 to 186, wherein said selective DOR antagonist is selected from the group consisting of benzylidenenaltrexone, naloxone, naltrexon, quadazocine, TIPPψ, diprenorphine, naltrindole, methylnaltrindole, N,N(Me)₂-Dmt-Tic-OH, SoRI-9409, naltriben, derivatives thereof and pharmaceutically acceptable salts and/or solvates thereof.

188. The cosmetic skin care composition of claim 187, wherein said selective DOR antagonist is selected from the group consisting of naltrindole, methylnaltrindole, N,N(Me)₂-Dmt-Tic-OH, SoRI-9409, naltriben and pharmaceutically acceptable salts and/or solvates thereof.

189. The cosmetic skin care composition of any one of claims 184 to 188, wherein said selective DOR antagonist is naltrindole or a pharmaceutically acceptable salt and/or solvate thereof.

190. The cosmetic skin care composition of any one of claims 184 to 186, wherein said selective DOR antagonist is a selective DOR₂ antagonist or a pharmaceutically acceptable salt and/or solvate thereof.

191. The cosmetic skin care composition of claim 190, wherein said selective DOR₂ antagonist is naltriben, TIPPψ (H-Tyr-TicPsi[CH(2)NH]Phe-Phe-OH) or a pharmaceutically acceptable salt and/or solvate thereof.

192. The cosmetic skin care composition of any one of claims 184 to 186, wherein said selective DOR antagonist is a selective DOR₁ antagonist or a pharmaceutically acceptable salt and/or solvate thereof.

193. The cosmetic skin care composition of claim 192, wherein said selective DOR₁ antagonist is benzylidenenaltrexone or a pharmaceutically acceptable salt and/or solvate thereof.

194. The cosmetic skin care composition of any one of claims 184 to 193, wherein said opioid receptor agonist is a mu opioid receptor (MOR) agonist, a DOR agonist or a pharmaceutically acceptable salt and/or solvate thereof.

195. The cosmetic skin care composition of claim 194, wherein the opioid receptor agonist is selected from the group consisting of SNC-80, BW373U86, DPI-287 and DPI-3290 and Met-enkephalin, (D-Ala²)deltorphin II, 7-spiroindanyloxymorphone, ADL-5859, BU-48, DADLE, deltorphin, (D-Pen²,D-Pen⁵)enkephalin (DPDPE), DPI-221, DSLET, Leu-enkephalin, RWJ-394,674, TAN-67, mitragyna speciosa, dihydromorphine, norbuprenorphine, N-phenethyl-14-ethoxymetopon, endomorphin, etonitazene, etorphine, fentanyl, methadone, morphine, normorphine and pentazocine, derivatives thereof or pharmaceutically acceptable salts and/or solvates thereof.

196. The cosmetic skin care composition of claim 194 or claim 195, wherein said opioid receptor agonist is a selective DOR agonist.

197. The cosmetic skin care composition of claim 196 wherein the selective DOR agonist is selected from the group consisting of SNC-80, BW373U86, DPI-287 and DPI-3290 or pharmaceutically acceptable salts and/or solvates thereof.

198. The cosmetic skin care composition of any one of claims 184 to 186, wherein the combination of a selective DOR antagonist and an opioid receptor agonist, or the combination of a selective DOR antagonist, an opioid receptor agonist and a selective ligand for a sensory receptor, comprises a mu-delta agonist-antagonist (MDAN) compound or pharmaceutically acceptable salts and/or solvates thereof.

199. The cosmetic skin care composition of claim 198, wherein said MDAN compound comprises a mu opioid receptor (MOR) agonist linked to a DOR antagonist by a linker comprising a backbone of at least 16 atoms, or pharmaceutically acceptable salts and/or solvates thereof.

200. The cosmetic skin care composition of claim 199, wherein said the MOR agonist linked to a DOR antagonist by a linker is oxymorphone or a derivative thereof.

201. The cosmetic skin care composition of claim 200, wherein said MDAN compound has a general formula (I): 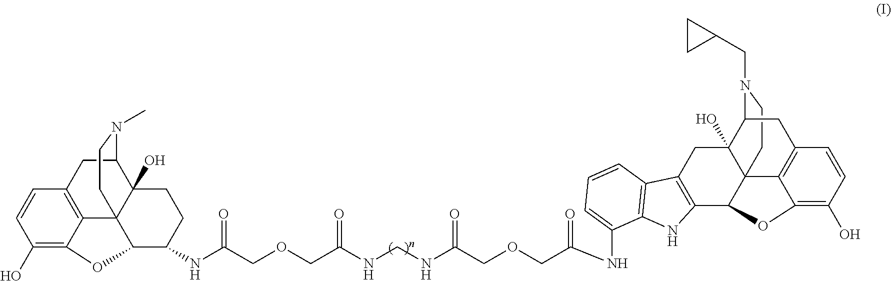
wherein n represents an integer of from 2 to 7, or pharmaceutically acceptable salts and/or solvates thereof.

202. The cosmetic skincare composition of any one of claims 184 to 201, wherein the composition is for enhancing the appearance or odor of the body by improving at least one property of skin selected from the group consisting of wrinkles, elasticity, atrophy, texture, radiance, skin colour, tone and pigmentation and making skin fair, skin renewal, rejuvenation, reduction of pores and controls oily or dry skin, and pleasant skin feelings.

203. The cosmetic skin care composition of any one of claims 184 to 202, wherein the composition is for stimulating growth and/or improvement of at least one of hair follicles or nails.

204. The cosmetic skin care composition of any one of claims 184 to 203, further comprising at least one selective ligand for a sensory receptor.

205. The cosmetic skin care composition of claim 204, wherein the sensory receptor comprises a taste receptor and/or an olfactory receptor on a skin cell.

206. The cosmetic skin care composition of claim 204 or claim 205, wherein the selective ligand is thujone or flufenamic acid.

207. A cosmetic skin care kit comprising:
(A) at least a first cosmetic skin care composition, the first cosmetic skin care composition comprising a selective DOR antagonist and a cosmetically acceptable adjuvant, diluent or carrier; and
(B) at least a second cosmetic skin care composition, the second cosmetic skin care composition comprising an opioid receptor agonist and/or a selective ligand for a sensory receptor, and a cosmetically acceptable adjuvant, diluent or carrier, and optionally
(C) instructions for the simultaneous, concomitant or sequential administration of the selective DOR antagonist of (A) and the opioid receptor agonist and/or the selective ligand for a sensory receptor of (B), to a subject in need thereof.

208. The cosmetic skin care kit of claim 207, wherein the selective DOR antagonist of (A) and the opioid receptor agonist and/or the selective ligand for a sensory receptor of (B) are for simultaneous or concomitant administration to the subject.

209. The cosmetic skin care kit of claim 207, wherein the selective DOR antagonist of (A) and the opioid receptor agonist and/or the selective ligand for a sensory receptor of (B) are for sequential administration to the subject.

210. The cosmetic skin care kit of claim 209, wherein the selective DOR antagonist of (A) is for administration to the subject before the administration of the opioid receptor agonist and/or the selective ligand for a sensory receptor of (B).

211. The cosmetic skin care kit of claim 210, wherein the selective DOR antagonist of (A) is for administration to the subject at least one minute before the administration of the opioid receptor agonist and/or the selective ligand for a sensory receptor of (B).

212. The cosmetic skin care kit of claim 211, wherein the selective DOR antagonist of (A) is for administration to the subject between about 5 minutes to about 15 minutes before the administration of the opioid receptor agonist and/or the selective ligand for a sensory receptor of (B).

213. The cosmetic skin care kit of claim 211, wherein the selective DOR antagonist of (A) is for administration to the subject from about one day to about two days before the administration of the opioid receptor agonist and/or the selective ligand for a sensory receptor of (B).

214. A method for screening selective ligands for a sensory receptor, comprising the steps of over-expressing a sensory receptor in epithelial cells, and screening selective ligands for the sensory receptor.

215. The method for screening according to claim 214, wherein the screening for selective ligands that are selective for the sensory receptor comprises measuring the activity of the sensory receptors after binding to the ligands that are selective for the sensory receptor.

216. The method for screening according to claim 214 or claim 215, wherein the sensory receptor is a taste receptor.
